(19) United States
(12) Patent Application Publication
(10) Pub. No.: US 2007/0248970 A1
(43) Pub. Date: Oct. 25, 2007

(54) COMPOSITIONS AND METHODS FOR DETECTING KERATOCONUS

(75) Inventors: YARON S. RABINOWITZ, Los Angeles, CA (US); Lijin Dong, Naperville, IL (US); Graeme J. Wistow, Silver Springs, MD (US)

Correspondence Address: KAREN S. CANADY;CANADY & LORTZ LLP, COMMERCE PLAZA, 11340 WEST OLYMPIC BLVD., SUITE 275, LOS ANGELES, CA, 90064, US

(73) Assignee: Eye Birth Defects Research Foundation, Los Angeles, CA (US)

(21) Appl. No.: 11/689,998

(22) Filed: Mar. 22, 2007

Related U.S. Application Data
(60) Provisional application No. 60/785,735, filed on Mar. 24, 2006.

(51) Int. Cl. C12Q 1/68; C07H 21/04; C12P 19/34

(52) U.S. Cl. 435/6; 435/91.2; 536/24.3

(57) ABSTRACT

The invention provides a method and assay kits for detecting keratoconus, including sub clinical keratoconus, in a specimen. The method comprises assaying a specimen of corneal epithelium for the presence of an expression product of the AQP5 gene. The invention further provides a novel gene, KC6, that exhibits cornea-specific expression, as well as KC6-related molecules.

This application claims the benefit of U.S. provisional patent application No. 60/785,735, filed Mar. 24, 2006, the entire contents of which are incorporated herein by reference. Throughout this application various publications are referenced. The disclosures of these publications in their entireties are hereby incorporated by reference into this application in order to describe more fully the state of the art to which this invention pertains. Several of these references are indicated by numerals in parentheses. A list of the corresponding reference citations can be found at the end of Examples 2 and 3.

### TECHNICAL FIELD OF THE INVENTION

The present invention relates generally to materials and methods for detecting keratoconus, including sub clinical keratoconus, in clinical specimens.

### BACKGROUND OF THE INVENTION

The concept of sub clinical keratoconus (KC), i.e. KC in the absence of clinical signs, sometimes referred to as ‘keratoconus fruste’ is now well established in the ophthalmic literature (1,2). This diagnosis often presents as a diagnostic dilemma and is assuming increasing importance as the number of refractive eye laser surgeries increase worldwide. It is commonly believed that the major cause of corneal ectasia after LASIK results from operating on patients with unrecognized sub clinical KC (3,4).

Modern videokeratography devices have significantly improved our ability to screen patients for patterns suggestive of sub clinical disease (5). However, reports of corneal ectasia developing after LASIK in patients with normal videokeratography patterns and adequate residual stromal bed thickness suggests there are patients with sub clinical KC that is so early that it remains undetectable even with videokeratography (6). A molecular genetic test to detect these at risk patients would significantly improve the safety of refractive surgery.

To ensure safe refractive surgery in all patients and to confirm the potential for development of KC in high risk populations for genetic counseling and linkage studies, there remains a need for a molecular genetic test to diagnose sub clinical KC.

### SUMMARY OF THE INVENTION

The invention provides a method for detecting keratoconus, including sub clinical keratoconus, in a specimen. The method comprises assaying a specimen of corneal epithelium for the presence of an expression product of the AQP5 gene. An absence or reduction of the AQP5 gene product is indicative of the presence of keratoconus. In one embodiment, the specimen comprises isolated RNA. Typically, the RNA is mRNA, and the assaying comprises polymerase chain reaction (PCR).

In one embodiment, the method further comprises assaying the specimen for a second gene product known to be present in corneal epithelium independent of the presence of keratoconus. The method can further comprise comparing the amount of expression product of the AQP5 gene to the amount of expression product of the second gene. The second gene can be any gene whose expression product is present in corneal epithelium and is not altered by the presence of keratoconus, clinical or sub clinical. Such a second gene can be used as a positive control and for comparison to AQP5 expression. One example of a second gene for this purpose is ESX-1. Another example is KC6, a novel gene described herein.

Also provided is an assay kit for detecting keratoconus, including sub clinical keratoconus, in a specimen. The kit comprises a pair of PCR primers that specifically amplify AQP5, and a set of written instructions for using the primers to perform the method of the invention. In a preferred embodiment, the PCR primers produce an amplicon that crosses two introns of the AQP5 gene, such as those having the nucleotide sequences TCCACTGACTCCCGCCGCACCAGC (SEQ ID NO: 2) and TCAGCGGGTGGTCAGCTCCATGGT (SEQ ID NO: 3).

The assay kit can further comprise a second pair of primers that specifically amplify a second gene known to be present in corneal epithelium independent of the presence of keratoconus. Typically, the second gene is ESX-1, or optionally, KC6. The second pair of PCR primers, for ESX-1, have the nucleic acid sequences AGGGCAAGAAGAGCAAGCAC (SEQ ID NO: 4) and AACTTGTAGACGAGTCGCCG (SEQ ID NO: 5). For KC6 (GenBank Accession No. NR_{—}002838), the second pair of primers have nucleic acid sequences selected from:

| | |
|---|---|
| AGAATCCAGGGGAAGATGAAGCAGC; | (SEQ ID NO: 6) |
| GAGGAAGCATAGGTTGAATGATCTG; | (SEQ ID NO: 7) |
| ATTGTGTATACATGGAGGTGGGATG; | (SEQ ID NO: 8) |
| and | |
| AGGTCAAGCAATCTAAGCTGCATAG. | (SEQ ID NO: 9) |

Alternatively, the invention provides an assay kit for detecting keratoconus, including sub clinical keratoconus, in a specimen comprising an oligonucleotide probe that specifically hybridizes with AQP5 and a set of written instructions for using the probe to perform the method of the invention. The kit can optionally comprise a further probe that specifically hybridizes with a second gene known to be present in corneal epithelium independent of the presence of keratoconus.

The invention additionally provides an isolated polynucleotide, KC6, having the nucleic acid sequence set forth herein. Also provided is an isolated polynucleotide primer that amplifies KC6, having a nucleic acid sequence selected from:

| | |
|---|---|
| AGAATCCAGGGGAAGATGAAGCAGC; | (SEQ ID NO: 6) |
| GAGGAAGCATAGGTTGAATGATCTG; | (SEQ ID NO: 7) |
| ATTGTGTATACATGGAGGTGGGATG; | (SEQ ID NO: 8) |
| and | |
| AGGTCAAGCAATCTAAGCTGCATAG. | (SEQ ID NO: 9) |

In addition, the invention provides probes that specifically hybridize under highly stringent conditions to the KC6 polynucleotide sequence shown above, fragments and complementary sequences of the KC6 polynucleotide that can be used as probes, as well as KC6 polynucleotides that encode KC6 polypeptides and antibodies that specifically bind KC6 polypeptides. These KC6 molecules can be used as a marker for corneal epithelium and corneal stem cells. KC6 regulatory sequences can be used to effect corneal-specific expression and to target delivery and expression of markers and therapeutic molecules to corneal tissues.

### BRIEF DESCRIPTION OF THE FIGURES

FIG. 1: Experiment 1, RT-PCR of 9 mm epithelial samples comparing myopia to keratoconus demonstrating an absence of AQP5 in the keratoconus samples, but the presence of ESX in both

FIG. 2: Experiment 2, Confirmation of the absence of AQP5 in 6 Keratoconus transplant buttons versus 2 normal controls (donor cornea scleral rim cornea from donor cornea(N4) and host corneal button from a patient with trauma (N1).

FIG. 3: Experiment 2, Densitometry ratios of AQP5/ESX 1 of Normals (N1-N5) versus keratoconus (K7), (N1—host cornea of patient with Fuchs Dystrrophy; N3-7 mm normal myopic epithelium and N2 and N5 corneal specimens from donor cornea scleral rims of donor corneas.

FIG. 4. A novel splice variant of the CRTAC1 gene from KC cornea.

FIG. 4A. Diagram of CRTAG1 gene structure showing the novel alternative splice pattern revealed by clones from KC cornea. Top line shows the canonical CRTAC1 gene with vertical blocks indicating exons. Bottom line shows the variant expressed in KC cornea with the three variant of alternative exons numbered 1a, 2a, 2b.

FIG. 4B. Predicted amino acid sequence of the variant CRTAC1 protein arising from the variant transcript (SEQ ID NO: 10). The variant N-terminal domain is boxed to show the regions that arise from the alternative exons 1a 2a, 2b. The remainder of the protein is the same as canonical CRTAC1.

FIG. 5: KC6, a novel gene from KC cornea. The six exons defined by cDNAs from KC cornea are shown approximately to scale together with alternative splicing patterns that have been observed so far. The arrows above the gene figure illustrate the three alternative splice forms identified. Block arrows below the gene diagram show the relative positions of primers used for RT-PCR. The sequence of alternative splice form 3, including sequence from all six exons has GenBank Accession No, NR_{—}002838.

FIG. 6: KC6 Expression is cornea-preferred.

FIG. 6A. RT-PCR detection of KC6 expression in epithelial samples of non-KC cornea (NC) and KC cornea. Upper panel shows results for intron-spanning KC6 specific primers from the 5′ region of the gene. Lower panel shows results for ELF3/ESX (ESX).

FIG. 6B. Expression of KC6 and ELF3/ESX (ESX) in human cornea (non-KC), retina, brain, kidney and heart. Panels KC6 3′ and 5′ show results for two different primer sets located near the 3′ and 5′ ends of the KC6 gene as shown in FIG. 5. Lower panel shows results for ESX.

FIG. 7: AQP5 expression is suppressed in KC cornea. RT-PCR of AQP5 and ELF3/ESX (ESX) for individual epithelial samples of KC and non-KC (NC) human cornea.

### DETAILED DESCRIPTION OF THE INVENTION

The present invention is based on the discovery and development of materials and methods for detecting and diagnosing keratoconus, including sub clinical keratoconus, in clinical specimens. This method provides a much-needed tool for screening patients for ophthalmic surgery and other clinical situations in which the detection of keratoconus is of value. The invention provides an assay kit for carrying out the method. Also provided is a novel gene specific to cornea, providing a marker for corneal epithelium and other cornea-related uses.

Definitions

All scientific and technical terms used in this application have meanings commonly used in the art unless otherwise specified. As used in this application, the following words or phrases have the meanings specified.

As used herein, “corneal epithelium” includes tissue from corneal buttons, epithelial rim and/or cornea scleral rim.

As used herein, an expression product of a gene is “reduced” in a specimen if there is at least a 4-fold reduction relative to the amount of that expression product present in a control specimen obtained from normal tissue, or relative to the expression product of a second gene (positive control) known to be expressed in corneal epithelium, regardless of keratoconus status (e.g., ESX-1). In some embodiments, the reduction is greater than 4-fold, such as 10-fold. Reductions of at least 32-fold have been observed.

As used herein, an “oligonucleotide probe” is an oligonucleotide having a nucleotide sequence sufficiently complementary to its target nucleic acid sequence to be able to form a detectable hybrid probe:target duplex under high stringency hybridization conditions. An oligonucleotide probe is an isolated chemical species and may include additional nucleotides outside of the targeted region as long as such nucleotides do not prevent hybridization under high stringency hybridization conditions. Non-complementary sequences, such as promoter sequences, restriction endonuclease recognition sites, or sequences that confer a desired secondary or tertiary structure such as a catalytic active site can be used to facilitate detection using the invented probes. An oligonucleotide probe optionally may be labeled with a detectable marker such as a radioisotope, a fluorescent moiety, a chemiluminescent moiety, an enzyme or a ligand, which can be used to detect or confirm probe hybridization to its target sequence. “Probe specificity” refers to the ability of a probe to distinguish between target and non-target sequences.

The term “nucleic acid”, “oligonucleotide” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally-occurring nucleotides.

As used herein, a “detectable marker” is a molecule attached to, or synthesized as part of a nucleic acid probe. This molecule should be uniquely detectable and will allow the probe to be detected as a result. These detectable moieties are often radioisotopes, chemiluminescent molecules, enzymes, haptens, or even unique oligonucleotide sequences.

As used herein, a “hybrid” or a “duplex” is a complex formed between two single-stranded nucleic acid sequences by Watson-Crick base pairings or non-canonical base pairings between the complementary bases.

As used herein, “hybridization” is the process by which two complementary strands of nucleic acid combine to form a double-stranded structure (“hybrid” or “duplex”). “Stringency” is used to describe the temperature and solvent composition existing during hybridization and the subsequent processing steps. Under high stringency conditions only highly complementary nucleic acid hybrids will form; hybrids without a sufficient degree of complementary will not form. Accordingly, the stringency of the assay conditions determines the amount of complementary needed between two nucleic acid strands forming a hybrid. Stringency conditions are chosen to maximize the difference in stability between the hybrid formed with the target and the non-target nucleic acid. Exemplary stringency conditions are described hereinbelow.

As used herein, “complementary” is a property conferred by the base sequence of a single strand of DNA or RNA which may form a hybrid or double-stranded DNA:DNA, RNA:RNA or DNA:RNA through hydrogen bonding between Watson-Crick base pairs on the respective strands. Adenine (A) ordinarily complements thymine (T) or Uracil (U), while guanine (G) ordinarily complements cytosine (C).

As used herein, the phrases “consist essentially of” or ”consisting essentially of” mean that the oligonucleotide has a nucleotide sequence substantially similar to a specified nucleotide sequence. Any additions or deletions are non-material variations of the specified nucleotide sequence which do not prevent the oligonucleotide from having its claimed property, such as being able to preferentially hybridize under high stringency hybridization conditions to its target nucleic acid over non-target nucleic acids.

One skilled in the art wilt understand that substantially corresponding probes of the invention can vary from the referred-to sequence and still hybridize to the same target nucleic acid sequence. This variation from the nucleic acid may be stated in terms of a percentage of identical bases within the sequence or the percentage of perfectly complementary bases between the probe and its target sequence. Probes of the present invention substantially correspond to a nucleic acid sequence if these percentages are from 100% to 80% or from 0 base mismatches in a 10 nucleotide target sequence to 2 bases mismatched in a 10 nucleotide target sequence. In preferred embodiments, the percentage is from 100% to 85%. In more preferred embodiments, this percentage is from 90% to 100%; in other preferred embodiments, this percentage is from 95% to 100%.

By “sufficientty complementary” or “substantiatly complementary” is meant nucleic acids having a sufficient amount of contiguous complementary nucteotides to form, under high stringency hybridization conditions, a hybrid that is stable for detection.

By “preferentially hybridize” is meant that under high stringency hybridization conditions oligonucleotide probes can hybridize their target nucleic acids to form stable probe target hybrids (thereby indicating the presence of the target nucleic acids) without forming stable probe:non-target hybrids (that would indicate the presence of non-target nucleic acids from other organisms). Thus, the probe hybridizes to target nucleic acid to a sufficiently greater extent than to non-target nucleic acid to enable one skilled in the art to accurately detect the presence of target sequence. Preferential hybridization can be measured using techniques known in the art and described herein.

As used herein, a “target nucleic acid sequence region” refers to a nucleic acid sequence present in the nucleic acid of an organism or a sequence complementary thereto, which is not present in the nucleic acids of other species. Nucleic acids having nucleotide sequences complementary to a target sequence may be generated by target amplification techniques such as polymerase chain reaction (PCR) or transcription mediated amplification.

As used herein, “a” or “an” means at least one, unless clearly indicated otherwise.

Probes of the Invention

The invention provides oligonucleotide probes that are specific for AQP5 and for KC6. The AQP5 gene encodes a protein having the amino acid sequence of SEQ ID NO: 1. The AQP5 coding sequence is shown in SEQ ID NO: 12.

KC6 has the amino acid sequence shown in SEQ ID NO: 10, and is encoded by the nucleotide sequence shown in SEQ ID NO: 11.

Oligonucleotides may be prepared using any of a variety of techniques known in the art. Oligonucleotide probes typically range in size from 10 to 100 nucleotides in length.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sutfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

Any polynucleotide may be further modified to increase stability. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Nucleotide sequences can be joined to a variety of other nucleotide sequences using established recombinant DNA techniques. For example, a polynucleotide may be cloned into any of a variety of cloning vectors, including plasmids, phagemids, lambda phage derivatives and cosmids. Vectors of particular interest include probe generation vectors. In general, a vector will contain an origin of replication functional in at least one organism, convenient restriction endonuclease sites and one or more selectable markers. Other elements will depend upon the desired use, and will be apparent to those of ordinary skill in the art.

Methods of the Invention

The invention provides a method for detecting keratoconus, including sub clinical keratoconus, in a specimen. The method comprises assaying a specimen of corneal epithelium for the presence of an expression product of the AQP5 gene. An absence or reduction of the AQP5 gene product is indicative of keratoconus. In one embodiment, the specimen comprises isolated RNA. Typically, the RNA is mRNA, and the assaying comprises polymerase chain reaction (PCR). Those skilled in the art are familiar with other methods of assaying and amplifying isolated nucleic acid molecules. The method can be carried out using primers that specifically amplify products of the AQP5 gene, or, alternatively, using probes that specifically hybridize to AQP5 and/or its products. Such probes are typically labeled with a detectable marker. In some embodiments, the expression product is a polypeptide, which is detected using an antibody that specifically binds AQP5. The antibody can be either polyclonal or monoclonal, and is typically labeled with a detectable marker.

In one embodiment, the method further comprises assaying the specimen for a second gene product known to be present in corneal epithelium independent of the presence of keratoconus. The method can further comprise comparing the amount of expression product of the AQP5 gene to the amount of expression product of the second gene. The second gene can be any gene whose expression product is present in corneal epithelium and is not altered by the presence of keratoconus, clinical or sub clinical. Such a second gene can be used as a positive control and/or for comparison to AQP5 expression. One example of a second gene for this purpose is ESX-1. Another example is KC6, a novel gene described herein whose expression is cornea-specific and abundant in both keratoconus and non-keratoconus specimens. As with AQP5, the second gene product can be detected using primers, probes and/or antibodies.

The specimens for use in the method are obtained using conventional means. Exemplary means are described in the Examples below. Preferably, the specimen comprises an epithelial sample of 1 mm or less. The clinician will appreciate the desirability of obtaining the minimal amount of tissue that will be acceptable to the patient and allow rapid epithelial regeneration with the least amount of pain or risk of infection. The patient or subject is typically human, although the method can be used with veterinary subjects as well.

In some embodiments, the target gene product (e.g., AQP5, ESX-1, KC6) is detected using both capture and detector probes. Accordingly, the target nucleic acid ultimately hybridizes with both capture probe(s) and detector probe(s). Although these two hybridization steps can be performed in any order, in one embodiment, detector probe hybridizes with the target nucleic acid first, after which the hybridized material is brought into contact with an immobilized capture probe. Following a wash, the dectector:target:capture combination is immobilized on a surface to which the capture probe has been bound. Detection of probe bound to target nucleic acid is indicative of presence of target sequence.

For use with an electrochemical sensor, such as a sensor array, the method comprises detection of current associated with binding of probe to target. In one embodiment, the capture probe is labeled with biotin and immobilized onto a surface treated with streptavidin. The detector probe can be detectably labeled, e.g. tagged with fluorescein, providing an antigen to which a horse radish peroxidase-labeled antibody binds. This peroxidase, in the presence of its substrate (typically, hydrogen peroxide and tetramethylbenzidine), catalyzes a well-characterized redox reaction and generates a measurable electroreduction current under a fixed voltage potential, thereby providing an electrochemical signal to detect presence of the target nucleic acid. Those skilled in the art are familiar with variations on this particular example that can be adapted to achieve the same objective.

Kits

For use in the diagnostic applications described herein, assay kits are also within the scope of the invention. Such kits can comprise a carrier, package or container that is compartmentalized to receive one or more containers such as vials, tubes, and the like, each of the container(s) comprising one of the separate elements to be used in the method. For example, the container(s) can comprise one or more primers as described herein, or a probe that is or can be detectably labeled. The kit can also include a container comprising a reporter-means, such as a biotin-binding protein, e.g., avidin or streptavidin, bound to a detectable label, e.g., an enzymatic, fluorescent, or radioisotope label.

The kit of the invention will typically comprise the container described above and one or more other containers comprising materials desirable from a commercial and user standpoint, including buffers, diluents, filters, needles, syringes, and package inserts with instructions for use. In addition, a label can be provided on the container to indicate that the composition is used for a specific application, and can also indicate directions for use. Directions and or other information can also be included on an insert which is included with the kit.

A typical assay kit for detecting keratoconus, including sub clinical keratoconus, in a specimen comprises a pair of PCR primers that specifically amplify AQP5, and a set of written instructions for using the primers to perform the method of the invention. In a preferred embodiment, the PCR primers produce an amplicon that crosses two introns of the AQP5 gene, such as those having the nucleotide sequences TCCACTGACTCCCGCCGCACCAGC (SEQ ID NO: 2) and TCAGCGGGTGGTCAGCTCCATGGT (SEQ ID NO: 3).

The assay kit can further comprise a second pair of primers that specifically amplify a second gene known to be present in corneal epithelium independent of the presence of keratoconus. Typically, the second gene is ESX-1, or optionally, KC6. The second pair of PCR primers, for ESX-1, have the nucleic acid sequences AGGGCAAGAAGAGCAAGCAC (SEQ ID NO: 4) and AACTTGTAGACGAGTCGCCG (SEQ ID NO: 5). For KC6 (GenBank Accession No. NR_{—}002838; SEQ ID NO: 11), the second pair of primers has nucleic acid sequences selected from:

| | |
|---|---|
| AGAATGCAGGGGAAGATGAAGCAGC; | (SEQ ID NO: 6) |
| GAGGAAGCATAGGTTGAATGATCTG; | (SEQ ID NO: 7) |
| ATTGTGTATACATGGAGGTGGGATG; | (SEQ ID NO: 8) |
| and | |
| AGGTCAAGCAATCTAAGCTGCATAG. | (SEQ ID NO: 9) |

Alternatively, the invention provides an assay kit for detecting keratoconus, including sub clinical keratoconus, in a specimen comprising an oligonucleotide probe that specifically hybridizes with AQP5 and a set of written instructions for using the probe to perform the method of the invention. The kit can optionally comprise a further probe that specifically hybridizes with a second gene known to be present in corneal epithelium independent of the presence of keratoconus.

KC6 Molecules

The invention additionally provides an isolated polynucleotide, KC6, having all or part of the nucleic acid sequence (SEQ ID NO: 11)

| | |
|---|---|
| 1 | tggagcctgt cagctgagca aaggagggaa actcaggatg |
| | gagaactcct ccccctccat |
| 61 | ccataagtgg cacaccactg gcctcctggt gttattaaag |
| | gaaaattgga aaagtttcca |
| 121 | gaagattaag cagaaagctt acaacttgca caagactctc |
| | aattttccag tggctctgac |
| 181 | agaaggataa atcttcggct gttttaacta aaatggacgg |
| | tcaagttggt atccatcgtg |
| 241 | aagctaaatg aatatctgga caggagtcag acaaagggaa |
| | ggagaatcca ggggaagatg |
| 301 | aagcagctgc aggcctgtct gctaccctat atccggggaa |
| | gagccagaaa ttgctgacca |
| 361 | tagagagtta cattgtccag cttttcctcc acgaaatgga |
| | gcagcaacaa aaggccagat |
| 421 | cattcaacct atgcttcctc aagtctgata acattagcgt |
| | ttctctcaga tatcagttca |
| 481 | agtaaggagt tgccaataga agcaaagatg ccaggaatca |
| | tcgtttttat ttcagtgtcc |
| 541 | ttacgaagac ctttgagcac aatggaacga tgaggcaaca |
| | ttaaatatta ctggaatggg |
| 601 | gtatgaaagc agctactaga aaatcctgca agaaaaatag |
| | atggagaaac tgagtgctaa |
| 661 | gagtcttgcc ttttagctgt caacagtttg aagatgaaac |
| | aaatctggaa cagctgattt |
| 721 | ctattgtggc agctattacc catttttgtt atgcatctgt |
| | ctcttcccag actataaatt |
| 781 | atttaaggtg tgagtcggag acctaggcat ttcttatggc |
| | cagaacctta agcaatgtct |
| 841 | gggatatagc agatataaca gactctcagc tattgcgtga |
| | attaaggaat gagcttgaga |
| 901 | aatactttta agtattttct tcctggaaaa gagactccaa |
| | ctgtcttttg tacagtgctt |
| 961 | gaaactcata ccattaatca ctctttgaga ctaaagctct |
| | gataacctga gtgaagaatt |
| 1021 | ggttacaagg acgttacaat gcaccatgct accttcagcc |
| | atttgtgctt ctgcataaat |
| 1081 | tgttttgacc gattagccaa tcagtcacca acaagaccca |
| | aaaggaatga gaatctaaac |
| 1141 | agattgtcac catttggtgg gtttgaacac caagaggata |
| | gagcctagag cattgtggtc |
| 1201 | tgctttaaga cttctgagag gcaataaggt aaggtctgaa |
| | aaaccaagag tcgggataaa |
| 1261 | caagtcaatt gtgtatacat ggaggtggga tgaataaaga |
| | ggaggagggg ttgatggaag |
| 1321 | aaaactgcac ccaaaattat cgtgtgtcaa aggctggctg |
| | tggagccgat cttacaacct |
| 1381 | caaataatgt gatcatacta tcactcagca taatttgtgc |
| | cccccagttt tgtatgtcta |
| 1441 | ggctgcttag gaaattgatg aataacagaa gatgccactt |
| | atcactgaga tgaaccaagt |
| 1501 | catcccagag ggagtactat ttgaagatgt gtggaacatt |
| | ctgtgaatat agaaaaggat |
| 1561 | cccataccat gattcaattc atgactgtgg ctagtagaga |
| | tccacatttc atgttttgct |
| 1621 | atccatggga tttaaatctg ctgtcactct ccttcctatg |
| | cagcttagat tgcttgacct |
| 1681 | aacattacaa ttaaagctta cataaatgct aaaaaaaaaa |
| | aaaaaa. |

Preferred polynucleotides comprise at least 15 consecutive nucleotides, preferably at least 30 consecutive nucleotides and more preferably at least 45 consecutive nucleotides, that encode a KC6 polypeptide. Polynucleotides that are fully complementary to any such sequences are also encompassed by the present invention. Polynucleotides may be single-stranded (coding or antisense) or doublestranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials. Portions of such KC6 polynucleotides can be useful as primers and probes for the amplification and detection of KC6 related molecules in tissue specimens.

Polynucleotides may comprise a native sequence (i.e., an endogenous sequence that encodes a KC6 polypeptide or a portion thereof) or may comprise a variant of such a sequence. Polynucleotide variants contain one or more substitutions, additions, deletions and/or insertions such that the immunogenicity of the encoded polypeptide is not diminished, relative to a native KC6 protein. Variants preferably exhibit at least about 70% identity, more preferably at least about 80% identity and most preferably at least about 90% identity to a podynucleotide sequence that encodes a native KC6 protein or a portion thereof.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, Mo. (1978) A model of evolutionary change in proteins—Matrices for detecting distant relationships. In Dayhoff, Mo. (ed.) Atlas of Protein Sequence and Structure, National Biomedical Research Foundation, Washington DC Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 411-17; Robinson, E. D. (1971) Comb. Theor. 11:105; Santou, N., Nes, M. (1987) Mol. Biol. Evol. 4:406-425: Sneath. P. H. A. and Sokal, R. R. (1973) Numerical Taxonomy the Principles and Practice of Numerical Taxonomy, Freeman Press, San Francisco, Calif.; Wilbur, W. J. and Lipman, D. J. (1983) Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native KC6 protein (or a complementary sequence).

Also provided is an isolated polynucleotide primer that amplifies KC6, having a nucleic acid sequence selected from:

| | |
|---|---|
| AGAATCCAGGGGAAGATGAAGCAGC; | (SEQ ID NO: 6) |
| GAGGAAGCATAGGTTGAATGATCTG; | (SEQ ID NO: 7) |
| ATTGTGTATACATGGAGGTGGGATG; | (SEQ ID NO: 8) |
| and | |
| AGGTCAAGCAATCTAAGCTGCATAG. | (SEQ ID NO: 9) |

In addition, the invention provides probes that specifically hybridize under highly stringent conditions to the KC6 polynucleotide sequence shown above, fragments and complementary sequences of the KC6 polynucleotide that can be used as probes, as well as KC6 polynucleotides that encode KC6 polypeptides and antibodies that specifically bind KC6 polypeptides. These KC6 molecules can be used as a marker for corneal epithelium and corneal stem cells. KC6 regulatory sequences can be used to effect corneal-specific expression and to target delivery and expression of markers and therapeutic molecules to corneal tissues.

Antisense and Inhibitory Nucleic Acid Molecules

The antisense molecules of the present invention comprise a sequence substantially complementary, or preferably fully complementary, to all or a fragment of a KC6 gene. Included are fragments of oligonucleotides within the coding sequence of a KC6 gene, and inhibitory nucleotides that inhibit the expression of KC6. Antisense oligonucleotides of DNA or RNA complementary to sequences at the boundary between introns and exons can be employed to prevent the maturation of newly-generated nuclear RNA transcripts of specific genes into mRNA for transcription. Antisense RNA, including siRNA, complementary to specific genes can hybridize with the mRNA for that gene and prevent its translation. The antisense molecule can be DNA, RNA, or a derivative or hybrid thereof. Examples of such derivative molecules include, but are not limited to, peptide nucleic acid (PNA) and phosphorothioate-based molecules such as deoxyribonucleic guanidine (DNG) or ribonucleic guanidine (RNG).

Antisense RNA can be provided to the cell as “ready-to-use” RNA synthesized in vitro or as an antisense gene stably transfected into cells which will yield antisense RNA upon transcription. Hybridization with mRNA results in degradation of the hybridized molecule by RNAse H and/or inhibition of the formation of translation complexes. Both result in a failure to produce the product of the original gene.

Both antisense RNA and DNA molecules and ribozymes of the invention may be prepared by any method known in the art for the synthesis of RNA molecules. These include techniques for chemically synthesizing oligonucleotides such as solid phase phosphoramidite chemical synthesis. Alternatively, RNA molecules may be generated by in vitro or in vivo transcription of DNA sequences encoding the antisense RNA molecule. Such DNA sequences may be incorporated into a wide variety of vectors with suitable RNA polymerase promoters such as T7 or SP6. Alternatively, antisense cDNA constructs that synthesize antisense RNA constructively or inducibly can be introduced into cell lines, cells or tissues.

DNA molecules may be modified to increase intracellular stability and half-life. Possible modifications include, but are not limited to, the addition of flanking sequences of the 5′ and/or 3′ ends of the molecule or the use of phosphorothioate or 2′ O-methyl rather than phosphodiesterase linkages within the backbone of the molecule. Other modifications include the use of chimeric antisense compounds. Chimeric antisense compounds of the invention may be formed as composite structures of two or more oligonucleotides, modified oligonucleotides, oligonucleosides and/or oligonucleotide mimetics. Such compounds have also been referred to in the art as hybrids or gapmers. Representative United States patents that teach the preparation of such hybrid structures include, but are not limited to, U.S. Pat. Nos. 5,700,922 and 6,277,603.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Antisense compositions of the invention include oligonucleotides formed of homopyrimidines that can recognize local stretches of homopurines in the DNA double helix and bind to them in the major groove to form a triple helix. See: Helen, C and Toulme, J J. Specific regulation of gene expression by antisense, sense, and antigene nucleic acids. Biochem. Biophys Acta, 1049:99-125, 1990. Formation of the triple helix would interrupt the ability of the specific gene to undergo transcription by RNA polymerase. Triple helix formation using myc-specific oligonucleotides has been observed. See: Cooney, M, et al. Science 241:456-459.

Antisense sequences of DNA or RNA can be delivered to cells. Several chemical modifications have been developed to prolong the stability and improve the function of these molecules without interfering with their ability to recognize specific sequences. These include increasing their resistance to degradation by DNases, including phosphotriesters, methylphosphonate, phosphorothioates, alpha-anomers, increasing their affinity for binding partners by covalent linkage to various intercalating agents such as psoralens, and increasing uptake by cells by conjugation to various groups including polylysine. These molecules recognize specific sequences encoded in mRNA and their hybridization prevents translation of and increases the degradation of these messages.

Antisense compositions including oligonucleotides, derivatives and analogs thereof, conjugation protocols, and antisense strategies for inhibition of transcription and translation are generally described in: Antisense Research and Applications, Crooke, S. and B. Lebleu, eds. CRC Press, Inc. Boca Raton Fla. 1993: Nucleic Acids in Chemistry and Biology Blackburn, G. and M. J. Gait, eds. IRL Press at Oxford University Press, Inc. New York 1990; and Oligonucleotides and Analogues: A Practical Approach Eckstein, F. ed., IRL Press at Oxford University Press, Inc. New York 1991; which are each hereby incorporated herein by reference including all references cited therein which are hereby incorporated herein by reference.

### EXAMPLES

The following examples are presented to illustrate the present invention and to assist one of ordinary skill in making and using the same. The examples are not intended in any way to otherwise limit the scope of the invention.

### Example 1

### Aquaporin 5: A Molecular Marker for Keratoconus

This example demonstrates the suppression of Aquaporin 5 (AQP5) in keratoconus (KC) corneas transplant buttons. In this experiment, both the surgeon and the molecular geneticist performing the molecular work knew the diagnosis of the specimens submitted for RT-PCR analysis.

i) Tissue Procurement

a) cDNA Library

Seven KC corneal host buttons approximately 7.5 mm in size were removed at the time of penetrating keratoplasty. A piece of tissue was sent for histopathology, and the rest immediately immersed in preservative (RNAlater, Ambion, Austin Tex.). All patients had moderate to advanced KC, with some having central corneal scarring. All patients had steep keratometry readings in excess of 60D and were contact lens intolerant. None of the patients had worn contact lenses for 3 months prior to undergoing corneal transplantation.

b) RT-PCR Experiments on Whole Corneas

Two additional corneal buttons were removed in a similar manner to that described above for RT-PCR experiments. One cornea had advanced KC with steep K readings in excess of 60D and the second cornea had posterior polymorphous dystrophy with normal K readings (less than 44D) and no evidence of KC either clinically or by videokeratography (2).

c) RT-PCR Experiments on Corneal Epithelial Samples

Three 9 mm samples of epithelium were obtained from 3 KC patients and 3 normal myopic patients at the time of photo-therapeutic keratectomy and photorefractive keratectomy respectively. The 3 KC patients had mild to moderate KC with the steepest K readings less than 48D in all three patients. Diagnosis was confirmed clinically and by videokeratography with each KC patient having an asymmetric bowtie pattern with skewed steep radial axis above and below the horizontal meridian (2). The three myopic patients had refractive errors from −6D to −9D and were soft contact lens wearers but had not worn their contact lenses for at least 2 weeks prior to the procedure. Epithelium was removed by scraping with a 69 beaver blade after anesthetizing the cornea with 2 drops of tetracaine.

ii) cDNA library construction and cDNA Sequencing and Bioinformatics has previously been described and will not be repeated here (8); see Example 3

iii) RT-PCR Detection of AQP5

Corneal samples were immediately stabilized in RNAlater after harvesting, and individually processed for total RNA isolation. Total RNA was isolated using the RNeasy Mini Kit (Qiagen, Valencia, Calif.) by following manufacture recommended procedures. Briefly, 30 μg or less of each corneal tissue was first homogenized in a guanidinium isothiocyanate containing solution. Subsequently, homogenized tissue suspension was added to a mini silicone column, and bound RNA was washed and eluted in 30 μl of deionized water. The concentration of total RNA was then determined by its optical density. Reverse transcriptase (RT) reactions were carried out using 50 ng total RNA and random hexamers with the Sensiscript RT Kit (Qiagen, Valencia, Calif.) by following manufacture-recommended procedures. Individual cDNA pools obtained from RT reactions were then served as templates for PCR detection of AQP5. The sequences of the primers for AQP5 detection were forward primer: TCCACTGACTCCCGCCGCACCAGC (SEQ ID NO: 2) and reverse primer TCAGCGGGTGGTCAGCTCCATGGT (SEQ ID NO: 3). Expected PCR product is 354 bp, corresponding to positions 517 to 870 within the AQP5 mRNA molecule, The amplicon crosses two introns of the AQP5 gene.

Results

The gene library constructed from 7 pooled KC corneal transplants buttons failed to detect any transcripts for Aquaporin 5 (AQP5), despite detecting over 4100 corneal genes and all the well known genes and markers for corneal epithelium (12).

RT-PCR of the KC and non-KC buttons confirmed this observation and showed the presence of epithelial marker ESX-1 in both specimens but and absence of AQP5 in the KC specimen which was present in the non-KC specimens.

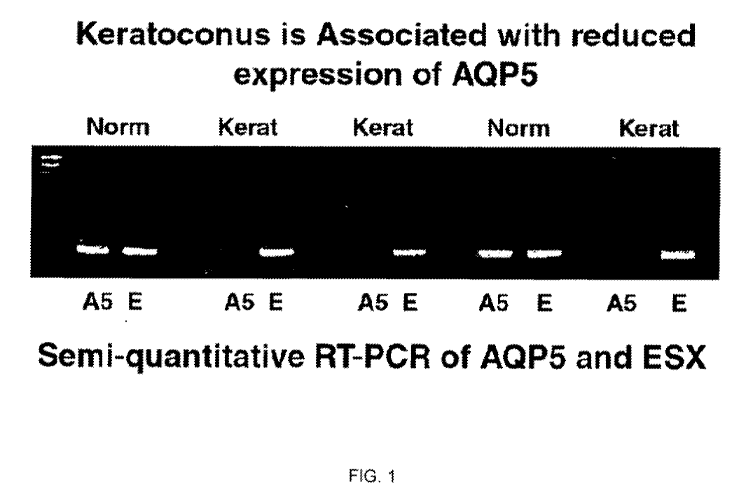

Follow up RT-PCR studies of 9 mm diameter epithelial specimens from KC patients undergoing PRK, once again demonstrated the absence of AQP5 but the presence of epithelial marker ESX-1, while the same size epithelial specimens from normal myopic patients revealed the presence of both ESX-1 and AQP5 in all specimens (FIG. 1).

### Example 2

### Aquaporin 5: A Molecular Marker For Detecting Sub Clinical Disease

This example demonstrates the suppression of Aquaporin 5 (AQP5) in keratoconus (KC) corneal transplant buttons, and use of this information to design a molecular genetic test for confirming the presence of sub clinical KC.

Material and Methods

Study Specimens and Tissue Procurement

Seven advanced KC(K readings greater than 60D) corneal buttons approximately 7.5 mm in diameter and two non KC buttons(one with trauma and one with Fuchs dystrophy) were immersed immediately in preservative (RNAlater, Ambion, Austin, Tex.) at the time of keratoplasty. 3 donor corneal scleral rim specimens were obtained from normal donor corneas and, one 7 mm diameter specimen of epithelium was also procured from a normal 6D myope at the time of PRK.

To test whether corneal epithelial samples alone would be adequate for diagnostic purposes we also compared three 9 mm epithelial specimens from myopic patients at the time of PRK to three 9 mm specimens from the patients with moderate KC (K reading less than 50D).

All patients signed a consent form approved by the Western Institutional Review Board (http://www.irb.com), allowing corneas to be used for the purposes of genetic research.

RNA preparation and expression detection by RT-PCR- Corneal buttons were immediately immersed in RNAlater (Ambion Inc., Cambridgeshire, United Kingdom) after collection, and later individually processed for total RNA extraction. During the RNA extraction, the corneal button was cut into small slices and immersed into liquid nitrogen in a tissue mortar. After liquid nitrogen evaporated, the corneal button slices were granulated immediately in the mortar. TRIzol Reagent (Invitrogen, Carlsbad, Calif. 92008, 1 ml TRIzol Reagent for 20-30 μg corneal tissue) was added and the tissue was further granulated and the tissue extract was then transferred to an Eppendorf tube. RNA was further extracted followed the manufacturer's recommended protocol of TRIzol Reagent. The concentration of total RNA is then determined by a NanoDrop ND-1000, a micro UV spectrophotometer using 1 μl volume of RNA sample.

Reverse transcription (RT) reactions are carried out using 50 ng total RNA, oligo dT primer, and SuperScript III RNase H-Reverse Transcriptase (Invitrogen, Carlsbad, Calif. 92008) by following the manufacturer recommended procedures. Individual cDNA sample obtained from RT reactions then serve as templates for PCR to analyze the AQP5 expression by standard procedures. To examine RNA quality and quantitatively measure the AQP5 expression, the expression of a house keeping gene, ESX-1, was referenced. ESX-1 is considered as a moderately abundant marker with steadily expression in corneal epithelium (13). PCR was performed under the standard procedure. Briefly, one microliter of cDNA was added to a mixture containing PCR buffer, 0.2 mM of each dNTP, 0.5 μM of both forward and reverse primers and 1 unit of Taq DNA polymerase (ABI, Foster City, Calif.). The PCR primers used for AQP5 detection were same as in experiment 1, and the PCR primers for ESX-1 detection were AGGGCAAGAAGAGCAAGCAC (forward) and AACTTGTAGACGAGTCGCCG (reverse). For best amplification results, these two PCR amplifications were conducted separately, with a 62° C. of annealing temperature with 40 cycles for AQP5, while a 55° C. of annealing temperature with 35 cycles for ESX-1. The RT-PCR products were separated and analyzed on a 1% agarose gel. Quantitative analysis was applied by a densitometry (Alpha Innotech Corp., San Leandro, Calif.) to measure the intensity of PCR bands and to calculate the ratio of AQP5 expression versus ESX-1 expression in selected specimens studied.

Measurements of AQP5 mRNA Abundance by Real Time PCR

Real-time PCR was also performed to measure the amount of mRNA quantitatively and the ratio of mRNA abundance of AQP5 versus EL3 was determined for each sample. The real time PCR method applied in our lab was TaqMan technology (ABI, Foster City, Calif.). The assay procedure was performed according to the manufacturer's protocol.

Results

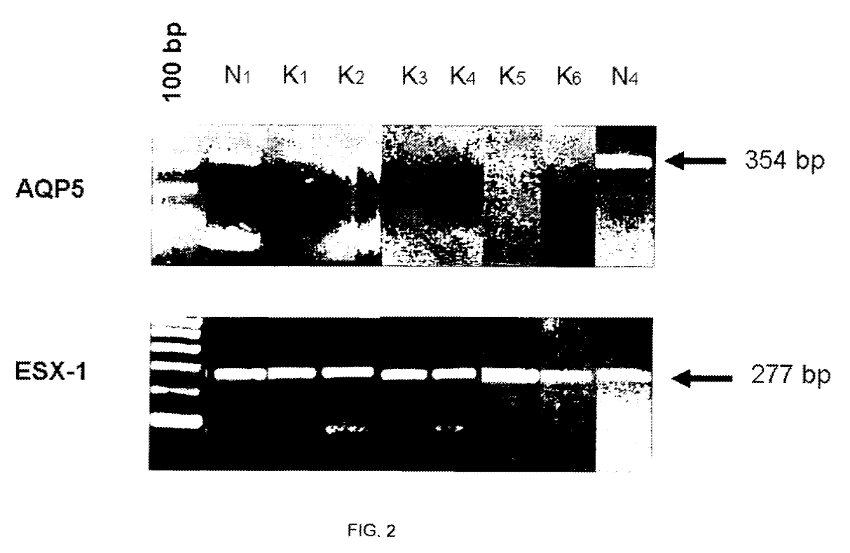

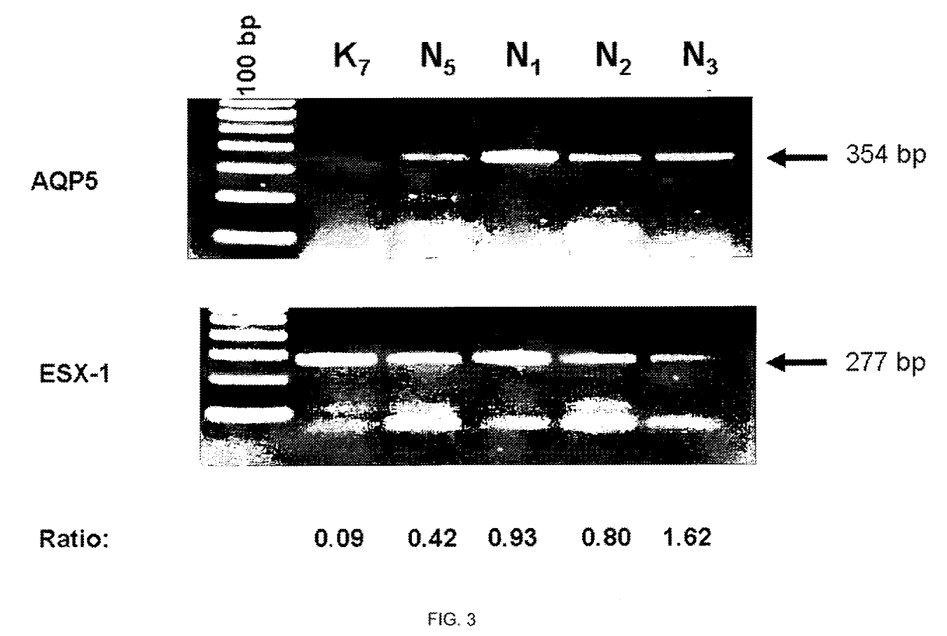

In all normal control specimens, the trauma and Fuchs dystrophy corneal buttons; the epithelial rims and 7 mm diameter epithelium from a normal myopic patient, the AQP5 band with the expected size of 354 bp was demonstrated (see FIG. 2, N1—donor epithelium from cornea scleral rim, N4—transplant button from patient with trauma), accompanied with an EL3 band with the expected size of 277 bp. Even with a mere 7 mm piece of epithelium good bands for each gene were clearly detected (see FIG. 3—N3). In 6 of the 7 KC specimens the AQP5 band was clearly absent. In one of the KC specimens, however, an extremely faint AQP5 band was observed (see FIG. 3—K7). For several samples the intensities of AQP5 band and EL3 bands were measured by a densitometry and the band intensity ratio of AQP5 versus EL3 was calculated. The intensity ratio for one KC sample (K7—FIG. 3) was 0.09, while the lowest ratio for “normal” was 0.42 (N5—FIG. 3—epithelium from cornea scleral rim), a more than 4-fold difference in expression of the two genes. For practical applications as a diagnostic test, we tested the AQP5 expression from the RNA extracted from the 7 mm diameter epithelial specimen only (FIG. 3—N3). The band intensity ratio of AQP5 versus EL3 was 1.62, an 18-fold difference in expression from the KC specimen (K7 in FIG. 3). Our preliminary results also show that epithelial specimens in patients moderate KC(K readings <50D) AQP5 is suppressed while it is not in the normal myopic PRK samples. A real time PCR method was established to measure the relative mRNA abundance of AQP5 versus EL3 quantitatively. All the samples, with RNA left, were tested by a real time PCR method and we obtained the similar results to RT-PCR. All the normal samples, with the exception of N2, demonstrated a higher value of ratio of AQP5 versus EL3, while all the KC samples revealed a lower ratio. The mean ratio of AQP5/EL3 for all normal specimens was 0.4, while the mean ratio for all KC specimens was 0.011, a 36 fold difference in the ratios of the expression of the two genes.

Discussion

We have previously reported the suppression of AQP5 in seven pooled KC corneas(7). In this report we tested 7 new independent KC samples with advanced disease in a different laboratory by a molecular geneticist who was blinded to the diagnosis. The presence of AQP5 was demonstrated in 7 new normal control specimens: the two non KC buttons, the 3 epithelial rims from donor corneas and the epithelial sample from a myopic PRK patient. In the 7 KC buttons AQP5 was suppressed. Additionally we have demonstrated the suppression of this gene in 3 epithelial samples of KC patients with moderate disease white it was present in the 3 normal control PRK specimens. This study demonstrates that not only are we able to use epithelial samples to make a molecular diagnosis of moderate KC but we are also able to quantify the ratios of the AQP5 gene compared to a normal control in a highly accurate and reproducible manner using real time PCR. This work lays the foundation for a potential highly accurate and reproducible test protocol which can be tested in a subpopulation of keratoconus ‘fruste’ or ‘suspect’ patients to develop a test for detecting sub clinical KC

For most genetic diseases the gold standard for a marker for disease detection is identifying a genetic mutation. This may be less than ideal from a diagnostic point of view, because different mutations occur in different families and not all disease causing mutations for a particular disorder is always identified. Detection of sub clinical KC by means of gene expression offers some interesting possibilities and a potentially better alternative than a genetic mutation since not all of KC has a genetic basis and if suppression of AQP5 occurs early, it has the potential to detect disease irrespective of whether it is of genetic or environmental origin (9, 10).

Furthermore, a specimen from the corneal epithelium is relatively easy to obtain and if it is small enough will create very little morbidity and regenerate very rapidly.

### REFERENCES

### Examples 1 and 2

1. Amsler M. Le keratoconus fruste au javal. Ophthalmologica 1938; 96:77-83.
2. Li X, Rabinowitz Y S, Rasheed K, Yang H. Longitudinal study of the normal eyes in unilateral keratoconus. Ophthalmology 2004; 111:44046.
3. Binder P S, Lindstrom R, Stulting R D, Donnenfeld E, Wu H, McDonnell P, Rabinowitz Y S, Keratoconus and corneal ectasia after LASIK. J Refract Surg 2005; 21:749-52
4. Randleman J B, Russell B, Ward M A, Thompson K P, Stulting R D. Risk factors and prognosis for corneal ectasia after LASIK. Ophthalmology 2003; 110:267-75.
5. Ambrosio R Jr, Klyce S D, Wilson S E. Corneal topographic and pachymetric screening of keratorefractive patients. J Refract Surg 2003; 19:24-29
6. Lifshitz T, Levy J, Klemperer I, Levinger S. Late bilateral keratectasia after LASIK in a low myopic patient. J Refract Surg. September-October 2005;21(5):494-6.
7. Agre P, Kozono D. Aquaporin water channels: molecular mechanisms for human diseases. FEBS Lett 2003: 555:72-78.
8. Rabinowitz Y S, Dong L, Wistow G. Gene expression profile studies of human Keratoconus cornea for NEIBank: A novel cornea expressed gene and the absence of transcripts for Aquaporin 5. Invest Ophthalmol Vis Sci 2005; 46:1239-46.
9. Rabinowitz Y S The genetics of keratoconus. Ophthalmol Clin North Am. December 2003;16(4):607-20. vii. Review.
10. Krachmer J H. Eye rubbing can cause keratoconus. Cornea 2004; 23:539-40.

### Example 3

### Gene Expression Profile Studies of Human Keratoconus Cornea and Identification of a Novel Cornea Expressed Gene

This example provides information increasing the database of genes expressed in human cornea and insights into the molecular basis of keratoconus (KC). A cDNA library was constructed from KC corneas harvested at keratoplasty and used for expressed sequence tag (EST) analysis. Data were analyzed using GRIST. Expression of selected clones was examined by RT-PCR. The five most abundant transcripts, represented by more than 60 clones each, were Keratin 12, BIGH3, decorin, ALDH3, and enolase 1; all known markers for cornea. Many other markers for epithelial, stromal and endothelial expressed genes were also present. One cluster of 6 clones came from an apparently novel gene (designated KC6) located on chromosome 18p12.3. RT-PCR of RNA from several human tissues detected KC6 transcripts only in cornea. In addition, no clones were observed for the usually prominent corneal epithelial cell marker aquaporin 5 (AQP5), a water channel protein. Semi-quantitative RT-PCR confirmed that expression of AQP5 is much tower in KC cornea than in non-KC cornea. KC6 is a novel gene of unknown function that shows cornea-preferred expression while the suppression of transcripts for AQP5 provides clear evidence of a molecular defect identified in KC.

Methods

Tissue Procurement

For library construction, seven Keratoconus (KC) corneal host buttons, approximately 7.5 mm in size, were removed with a Barron Hessburg trephine at the time of penetrating keratoplasty. A piece of tissue was sent for histopathology and the rest was immediately immersed in RNAlater (Ambion, Austin Tex.). The procedure for obtaining the tissues was within the tenets of the Declaration of Helsinki. Prior to corneal transplants each patient not only consented to have their corneas removed for therapeutic purposes but also signed an additional consent form approved by the Western IRB (http.//www.irb.com) allowing their corneas to be used for genetic research. Two additional corneas, one with keratoconus and one essentially normal cornea with mild endothelial polymorphous dystrophy were collected in the same way and used in RT-PCR experiments. In addition, 9 mm diameter samples of normal (myopic) and KC corneal epithelium were obtained from photorefractive keratectomy (PRK) or phototherapeutic keratectomy (PTK) and were also used for RT-PCR.

cDNA Library Construction

Seven KC corneal buttons were pooled and total RNA was extracted using RNAzol (Tel-Test Inc., Friendswood, Tex.). 40 μg of total RNA was used for cDNA synthesis. Poly(A)+ RNA was prepared using an oligo-dT cellulose affinity column.

Oligo-dT primed cDNA was synthesized at Bioserve Biotechnology (Laurel, Md.) using the Superscript 11 system (Invitrogen, Carlsbad Calif.), as described previously [6]. The cDNA was run over a resin column (Sephacryl S-500 HR; Invitrogen) to fractionate cDNA larger than 500 bp. The first two 35-μL fractions, containing cDNA, were directionally cloned in cloned in Not I/Sal I sites in the pCMVSPORT6 vector (Invitrogen) to make a cDNA sublibraries with code designation od and oe.

cDNA Sequencing and Bioinformatics

Methods for sequencing and bioinformatics analysis are described in detail elsewhere [6, 26]. Briefly, randomly picked clones were sequenced from the 5′ end at the NIH Intramural Sequencing Center (NISC). GRIST (Grouping and Identification of Sequence Tags) was used to analyze and assemble the data, and to display the results in Web page format [26]. Clusters of sequences were also examined using SeqMan II (DNAstar, Madison, Wis.) to check assembly of clusters and to examine alternative transcripts. Sequences were also searched through genome resources at the National Institutes of Health (http://www.ncbi.nlm.nih.gov/) and the University of California at Santa Cruz (http://genome.ucsc.edu/).

RT-PCR Detection of AQP5 and KC6 Transcripts

Corneal samples were immediately stabilized in RNAlater after harvesting, and individually processed for total RNA isolation. Total RNA was isolated using the RNeasy Mini Kit (Qiagen, GmbH, Germany) by following manufacture recommended procedures. Briefly, 30 mg or less of each corneal tissue was first homogenized in a guanidinium isothiocyanate containing solution. Subsequently, homogenized tissue suspension was added to a mini silicone column, and bound RNA was washed and eluted in 30 μl of deionized water. The concentration of total RNA was then determined by its optical density. Reverse transcriptase (RT) reactions were carried out using 50 ng total RNA and random hexamers with the Sensiscript RT Kit (Qiagen, GmbH, Germany) by following manufacture-recommended procedures. Individual cDNA pools obtained from RT reactions were then served as templates for PCR detection of AQP5 and KC6 expression. The sequences of the primers for AQP5 and KC6 detection were the following: (1)

| | |
|---|---|
| (1) AQP5: | |
| TCCACTGACTCCCGCCGCACCAGC; | (SEQ ID NO: 2) |
| AQP5 reverse: | |
| TCAGCGGGTGGTCAGCTCCATGGT; | (SEQ ID NO: 3) |
| (2); KC6 5′ forward: | |
| AGAATCCAGGGGAAGATGAAGCAGC; | (SEQ ID NO: 6) |
| KC6 5′ reverse: | |
| GAGGAAGCATAGGTTGAATGATCTG; | (SEQ ID NO: 7) |
| (3) KC6 3′ forward: | |
| ATTGTGTATACATGGAGGTGGGATG; | (SEQ ID NO: 8) |
| KC6 3′ reverse; | |
| AGGTCAAGCAATCTAAGCTGCATAG. | (SEQ ID NO: 9) |

Results and Discussion

Initial quality control sequencing of the od and oe sublibraries showed they were very similar in composition. Subsequently, approximately equal numbers of clones were sequenced from both sublibraries and the data were pooled. The combined od/oe libraries yielded a total of 7680 sequence reads of average length over 600 bp. The data contained about 2% contamination with mitochondrial genome sequences and less than 1% rRNA clones. After analysis by GRIST, 4090 clusters of clones, each potentially representing an individual transcribed gene, were identified. This is the largest set of cornea derived clones currently available and can be viewed in its entirety on the NEIBank website (http://www.neibank.nei.nih.gov).

Abundantly Expressed Genes

Table 1 shows the most abundant transcripts, genes represented by 8 or more cDNA clones. The five most abundant clones are all known corneal markers, Keratin 12, BIGH3, decorin, ALDH3, and enolase 1 [2, 10, 27-32]. Genes expressed in all three regions of the cornea are represented, including keratins 12 and 3 from the epithelium, decorin and keratocan from the stroma [33] and “ovary-specific protein” from the endothelium [11]. Several other keratins and proteoglycans known to be important in cornea are also abundant, including keratins 3 and 5, keratocan and lumican. Previously, in a specific effort to examine changes in gene expression in KC, microarray analysis was used to compare expression of 5600 genes using cDNA from KC and myopic corneas [19]. In this study, increased relative expression of keratins 6 and 13 was noted in the KC samples, but these are both absent from the cDNA library collection.

TABLE 1
The most abundantly represented genes in the KC cDNA library.
Genes represented by eight or more cDNAs are shown,
along with a representative GenBank entry and the number of clones
seen in the EST analysis.

| Gene Name | GenBank ID | # Clones |
|---|---|---|
| K12 keratin | D78367 | 283 |
| transforming growth factor-beta induced gene | M77349 | 101 |
| product (BIGH3) | | |
| decorin variant A | AF138300 | 101 |
| aldehyde dehydrogenase type III (ALDHIII) | M74542 | 95 |
| enolase 1, (alpha) (ENO1) | NM_001428 | 61 |
| TRPM-2/clusterin | M64722 | 46 |
| keratin 3 (KRT3), | NM_057088 | 35 |
| eukaryotic translation elongation factor 1 alpha | NM_001402 | 32 |
| 1 (EEF1A1) | | |
| keratin type II (58 kD) | M21389 | 31 |
| ferritin heavy chain subunit | AF088851 | 29 |
| keratan sulfate proteoglycan | AF063301 | 25 |
| NAD(P)H: menadione oxidoreductase | J03934 | 23 |
| transmembrane protein (THW gene) | AJ251830 | 23 |
| angiopoietin-like factor (CDT6), | NM_021146 | 22 |
| aldehyde dehydrogenase 1 family, member A1 | NM_000689 | 21 |
| (ALDH1A1) | | |
| lumican (LUM), | NM_002345 | 19 |
| prosaposin | D00422 | 18 |
| lipocortin II | D00017 | 17 |
| prostaglandin D2 synthase | NM_000954 | 13 |
| polyubiquitin UbC | AB009010 | 13 |
| tripartite motif-containing 29 (TRIM29) | NM_012101 | 13 |
| ASPIC (acidic secreted protein in | AJ276171 | 12 |
| cartilage)/CRTAC1 | | |
| collagen, type XVII, alpha 1 (COL17A1) | NM_000494 | 12 |
| apolipoprotein D | J02611 | 12 |
| transketolase (tk) | L12711 | 12 |
| paired box gene 6 (PAX6) | NM_001604 | 12 |
| ribosomal protein L4 | D23660 | 12 |
| pyruvate kinase, muscle (PKM2) | NM_002654 | 11 |
| desmoplakin | J05211 | 11 |
| HSP27 | AB020027 | 11 |
| gelsolin | NM_000177 | 11 |
| GAPDH | AY007133 | 11 |
| connexin 43 (GJA1, Cx43) | M65188 | 11 |
| glutamine synthase | X59834 | 10 |
| actin, gamma 1 (ACTG1), | NM_001614 | 10 |
| transmembrane protein BRI (BRI) | AF152462 | 10 |
| lactate dehydrogenase A (LDHA) | NM_005566 | 10 |
| LOC283120 (LOC283120) | XM_208516 | 9 |
| Hsp89-alpha-delta-N | AF028832 | 9 |
| SERPINB5 | NM_002639 | 9 |
| aspartate beta-hydroxylase (ASPH) | NM_004318 | 9 |
| calcium-activated chloride channel-2 | AF043977 | 9 |
| (hCLCA2) | | |
| desmoglein 1 (DSG1) | AF097935 | 9 |
| Ewings sarcoma EWS-Fli1 oncogene | AF327066 | 9 |
| non-integrin laminin-binding protein | M36682 | 9 |
| connexin 26 (GJB2) gene | AF479776 | 8 |
| DEAD (Asp-Glu-Ala-Asp) box polypeptide 5 | NM_004396 | 8 |
| (DDX5) | | |
| MEN1 region clone epsilon/beta | AF001893 | 8 |
| Syndecan-1 (SDC-1) | AJ551176 | 8 |
| ribosomal protein L3 (RPL3) | NM_000967 | 8 |
| ovary-specific acidic protein | AF329088 | 8 |

Another marker for epithelial cells that appears in the top 50 cDNAs is desmoglein 1 (DSG1), which is surprisingly abundant. The current version of Unigene contains only 35 ESTs for this gene (Hs.2633) from all sources, mostly from cell lines or tumors. In the KC cornea collection alone there are 9 clones for DSG1 plus another group of 4 ESTs located just 3′ to the gene in the human genome that are most likely derived from longer 3′ UTRs for the same gene. DSG1 is a calcium-binding transmembrane glycoprotein of the cadherin superfamily that is a component of desmosomes and is associated with the most differentiated cells within the cutaneous epithelium [34]. A single clone for the relate DSG2 is also present. The previous microarray analysis of KC cornea found expression of a third member of the family, DSG3, to be elevated in KC compared with myopic cornea [19], although again no clones for this gene are found in the cDNA collection. Such differences might be explained by cross-hybridization of related genes in the array experiment or insufficiently deep sequencing of the library to detect rare transcripts.

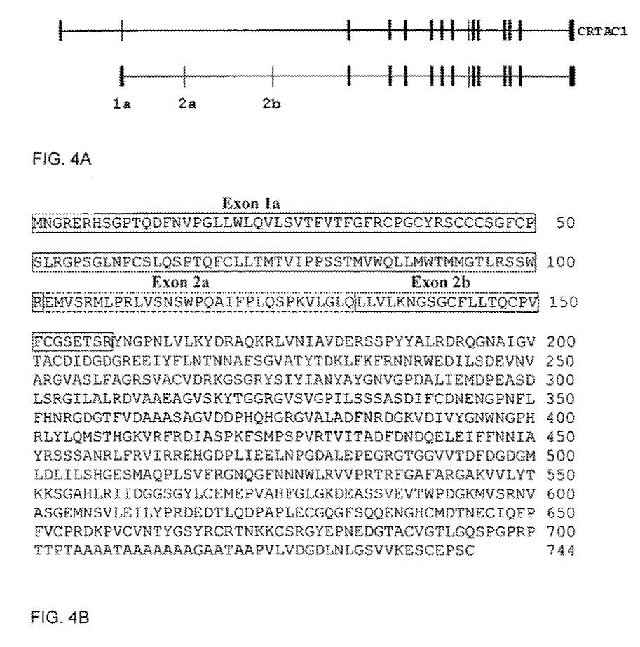

Among the most abundant cDNAs from KC cornea are those for cartilage acidic protein 1 (CRTAC1) also known as acidic secreted protein in cartilage 1 (ASPIC1) or CEP-68. This is a protein associated with the differentiated state of chondrocytes [35]. Six of the twelve clones are apparently full length and of these two make use of an alternative promoter, giving a variant 5′ end, and also include two novel alternatively spliced exons (FIG. 4). As a result, the predicted protein sequence resulting from these transcripts has a different, longer N-terminal domain containing several cysteine residues. Part of this alternative domain actually makes use of an alternative reading frame in a common part of what is the second exon of canonical CRTAC1. Since the function of the protein in chondrocytes is unclear and, since neither the known nor novel N-terminal domains have obvious similarity to other functional domains, it is not possible to predict what effects this might have. However, the abundance of this gene in KC cornea, with its problems of stromal thinning, suggests that it is worthy of further investigation, although the CRTAC1 gene is located on chromosome 10q24.2, which is not close to any mapped loci for KC.

Collagens

Collagen is a major structural component of the cornea and several clinical reports suggest an association between keratoconus and connective tissue disorders that may involve defects in collagen [36-40]. Table 2 lists the ten collagen genes represented in the KC cornea analysis. Two that are abundant in this collection, collagen 17A1 (12 clones) and collagen 12A1 (4 clones), and two represented by single clones, collagen 18A1 and collagen 21A1, have not previously been described in the cornea. The BodyMap data for normal corneal epithelium contains an additional collagen, 5A3, that is absent from the KC dataset. The KC dataset also lacks clones for collagens 2,7 and 8, which have been identified in normal cornea by immunochemical and peptide analysis [41] or for collagens 4A1, 11A1 or 16A1 that were identified by hybridization of cDNA library-derived probes to a commercial microarray [42]. Some of all of these differences could be due to insufficiently deep sequencing of the KC cornea cDNA library to detect some rare transcripts, to errors in identification in earlier analyses or to actual differences in the collagen expression profile of KC. All these differences will require further study to determine their significance.

TABLE 2
Collagen genes identified in the KC cornea cDNA library.

| Gene Description | GenBank | # Clones |
|---|---|---|
| collagen, type XVII, alpha 1 | NM_000494 | 12 |
| collagen, type I, alpha 2 | NM_000089 | 6 |
| collagen, type V, alpha 2 | J04478 | 4 |
| collagen, type XII, alpha 1 | NM_004370 | 4 |
| collagen, type VI, alpha 2 | AY029208 | 3 |
| collagen, type IV, alpha 3 | NM_000091 | 1 |
| collagen, type IV, alpha 6 | NM_033641 | 1 |
| collagen, type VI, alpha 3 | NM_004369 | 1 |
| collagen, type XXI, alpha 1 | AF330693 | 1 |

Transcription-Related Genes

Transcription factors play a central role in the differentiation and normal function of all tissues. Table 3 lists the most abundant (those represented by more than one cDNA clone) of the many transcription factors and transcription related proteins (broadly defined according to GO (Gene Ontology) annotation [43]) that have been identified in this study. It may be surprisingly to see that a prominent corneal marker, the glycolytic enzyme α-enolase, leads this list. α-enolase has been associated with transcription because a variant of this multifunctional protein that arises from an alternative transcript apparently has a role in control of expression of c-myc [44]. It seems unlikely that the high abundance of α-enolase in cornea has any direct association with transcription, and the α-enolase cDNAs in the collection show no evidence of alternative splicing; however, the abundance of α-enolase in cornea is also much greater than would be expected for a purely enzymatic role. Interestingly, α-enolase also serves as an abundant lens crystallin in some vertebrate species [45], suggesting that is may have the capacity to act in a structural or even protective role in tissues exposed to light. Indeed, another enzyme, ALDH3, is similarly very abundant in cornea, while other members of the ALDH superfamily also serve as lens crystallins in both vertebrates and invertebrates [46-50], suggesting that certain enzymes are able to play roles beyond their involvement in catalysis.

TABLE 3
Transcription factors represented by more than one cDNA clone in the current
KC cornea collection.

| Gene Description | GenBank | # Clones |
|---|---|---|
| enolase 1, (alpha) | NM_001428 | 61 |
| TRIM29 | NM_012101 | 13 |
| Pax6 | NM_001604 | 12 |
| ELF3/ESX | AF110184 | 6 |
| galectin 8 | L24123 | 6 |
| CBX3 | NM_016587 | 5 |
| ID1 | NM_002165 | 5 |
| ribosomal protein L7 | NM_000971 | 5 |
| basic transcription factor 3 | AB062126 | 4 |
| nuclear receptor coactivator 4 | NM_005437 | 4 |
| proteasome 26S subunit, ATPase, 5 | AF035309 | 4 |
| retinoblastoma 1 | L41870 | 4 |
| zinc finger protein 9 | M28372 | 4 |
| bromodomain adjacent to zinc finger domain, 2B | AB032255 | 3 |
| DNA-binding protein B/nuclease sensitive element binding protein 1 | L28809 | 3 |
| ERCC3 | M31899 | 3 |
| general transcription factor IIH, polypeptide 1, 62 kDa | BT007704 | 3 |
| heterogeneous nuclear ribonucleoprotein R | AF000364 | 3 |
| HMG-14 (High-mobility group nucleosome binding domain 1 | NM_004965 | 3 |
| HP1-BP74 | AF113534 | 3 |
| Kruppel-like factor 4 (gut) | AF105036 | 3 |
| Kruppel-like factor 5 (intestinal) | AF132818 | 3 |
| nuclear receptor coactivator 3 | AF012108 | 3 |
| O-sialoglycoprotein endopeptidase APEX nuclease 1 | D13370 | 3 |
| protein kinase, cAMP-dependent, regulatory, type I, alpha | M18468 | 3 |
| ribosomal protein S10 | AF521882 | 3 |
| splicing factor 3b, subunit 2, 145 kDa | XM_290506 | 3 |
| transcription elongation factor A (SII), 1 | X73534 | 3 |
| zinc finger protein 36, C3H type-like 1 | NM_004926 | 3 |
| zinc finger protein 67, hcKrox | AF007833 | 3 |
| ALL1 fused gene from 5q31 | L80196 | 2 |
| apoptotic chromatin condensation inducer in the nucleus | AF124726 | 2 |
| aryl hydrocarbon receptor | L19872 | 2 |
| basic helix-loop-helix domain containing, class B, 2 | AB004066 | 2 |
| CCR4-NOT transcription complex, subunit 7 | NM_013354 | 2 |
| checkpoint suppressor 1 | NM_005197 | 2 |
| chromodomain helicase DNA binding protein 3 | AF006515 | 2 |
| CXXC1 CpG binding protein | AJ132339 | 2 |
| EHF/ESE3 ets homologous factor | AF203977 | 2 |
| enhancer of zeste homolog 1 | D84064 | 2 |
| ERCC5 | NM_000123 | 2 |
| FOS-like antigen 2 | NM_005253 | 2 |
| high mobility group nucleosomal binding domain 3 | AY043282 | 2 |
| High mobility group protein 1 (HMG-1) | NM_002128 | 2 |
| HIV-1 Tat interactive protein, 60 kDa | NM_006388 | 2 |
| influenza virus NS1A binding protein | AF161553 | 2 |
| interferon regulatory factor 6 | NM_002229 | 2 |
| jun B proto-oncogene | NM_002229 | 2 |
| KIAA0194 protein heme-regulated initiation factor 2-alpha kinase | AF147094 | 2 |
| KIAA0669 gene product | AF201290 | 2 |
| leucine rich repeat (in FLII) interacting protein 1 | AF130054 | 2 |
| Meis1, myeloid ecotropic viral integration site 1 homolog | NM_002398 | 2 |
| MYST histone acetyltransferase 2 | AF140360 | 2 |
| NRAS-related gene | NM_007158 | 2 |
| nuclear factor (erythroid-derived 2)-like 2 | NM_006164 | 2 |
| paired mesoderm homeo box 1 | NM_006902 | 2 |
| period homolog 3 | AB047686 | 2 |
| polymerase (RNA) II (DNA directed) polypeptide B, 140 kDa | AF055028 | 2 |
| polymerase (RNA) II (DNA directed) polypeptide C, 33 kDa | NM_002694 | 2 |
| PR domain containing 4 | AF144757 | 2 |
| RNA-binding region (RNP1, RRM) containing 2 | L10910 | 2 |
| SAM and SH3 domain containing 1 | AJ507735 | 2 |
| scaffold attachment factor B | NM_002967 | 2 |
| signal transducer and activator of transcription 1, 91 kDa | BT007241 | 2 |
| splicing factor, arginine/serine-rich 11 | NM_004768 | 2 |
| sterol regulatory element binding transcription factor 1 | NM_004176 | 2 |
| suppressor of Ty 6 homolog | NM_003170 | 2 |
| TAR DNA binding protein | NM_007375 | 2 |
| T-box 2 | NM_005994 | 2 |
| TEA domain family member 3 | AF142482 | 2 |
| TRAF and TNF receptor associated protein | NM_016614 | 2 |
| transcriptional adaptor 3 (NGG1 homolog)-like | AF069733 | 2 |
| tubby like protein 4 | AF219946 | 2 |
| U2(RNU2) small nuclear RNA auxiliary factor 1 | AL832665 | 2 |
| upstream binding protein 1 (LBP-1a) | AK027177 | 2 |
| XPC | D21089 | 2 |
| ZNF593 | NM_015871 | 2 |

However, a large number of KC cornea cDNAs unambiguously represent transcription factors. Among the most abundant cDNAs for proteins with a well-established role in gene expression is Pax6, one of the key determinants of eye development [51]. Pax6 is able to exert tissue-specific effects through alternative splicing of the gene transcript that gives rise to different versions of the DNA binding domain in the protein. Indeed, the alternatively spliced exon 5a is used in half of the human KC cornea clones, which is consistent with previous observations which showed approximately equal levels of Pax6 mRNA with and without the alternative exon in bovine cornea [52]. Interestingly, four out of six apparently full-length Pax6 clones from KC cornea come the far upstream “A” promoter, which has previously been shown to be used primarily in cerebellum [53]. This may indicate some tissue preference in the expression mechanism for this important gene in cornea.

Many other transcription factors are represented and several have specific roles in epithelial tissues. One of these, ELF3 (also known as ESX, ERT, EPR-1, or ESE-1), is represented by 6 cDNA clones. ELF3 is an ets domain transcription factor specific to epithelial cells and is associated with terminal differentiation in the epidermis [54]. A second member of the same superfamily, EHF (also known as ESE-3 or ESEJ), is represented by 2 clones, while the collection contains a third clone that probably comes from a longer 3′UTR of the same gene. EHF is also associated with epithelial cells but has different target gene specificity from ELF3 [55]. Three other ets domain factors, ETV4, ETV5 and ERF are represented by single clones. ZFP67/hcKrox, a zinc finger protein that plays a rote in expression of type 1 collagen genes and has an important rote in epidermal differentiation [56], is represented by 3 clones. The forkhead domain protein FOXC1, which is the locus for various glaucoma phenotypes including primary congenital glaucoma, autosomal dominant iridogoniodysgenesis anomaly, and Axenfeld-Rieger anomaly [57], is represented by one clone.

VSX1, a transcription factor of the retina and the focus of a form of KC associated with retina anomalies [14], is not represented in the KC collection, but this could certainly be due to insufficiently deep sequencing to detect rare transcripts. Among the sequence collections from other NEIBank human eye cDNA libraries, only one VSX1 clone is found and that is in retina.

Apoptosis

Apoptosis, or programmed cell death, appears to be elevated in KC compared with normal donor corneas or those with stromal dystrophies [58]. Table 4 lists the apoptosis-related transcripts identified in this study, some of which are present at high levels. Clusterin is highly expressed in cornea and other eye tissues and, among many other possible roles, has been implicated in protection from apoptosis, although its role in cornea and elsewhere is not clear [32]. The second most abundant protein represented by cDNAs in this list is PERP (also known under several aliases including TP53 apoptosis effector, THW and PIGPC1). PERP is a tetraspanin protein of the plasma membrane that is a target gene for p53 and is highly up-regulated during p53-mediated apoptosis [59]. Other related tetraspanins that are also associated with apoptosis, EMP-1 and EMP-2 [60], are also present in the KC dataset.

TABLE 4
Genes associated with apoptosis identified in the KC cornea cDNA library.

| Gene Description | GenBank | # Clones |
|---|---|---|
| clusterin/apolipoprotein J | M64722 | 46 |
| p53-induced protein PIGPC1 (THW) | AJ251830 | 23 |
| epithelial membrane protein 1 | NM_001423 | 7 |
| serum/glucocorticoid regulated kinase | AF153609 | 4 |
| BCL2/adenovirus E1B 19 kDa interacting protein 3-like | AF067396 | 3 |
| caspase 6, apoptosis-related cysteine protease | NM_001226 | 3 |
| programmed cell death 4 (neoplastic transformation inhibitor) | NM_014456 | 3 |
| apoptotic chromatin condensation inducer in the nucleus | AF124726 | 2 |
| aryl hydrocarbon receptor | L19872 | 2 |
| BCL2/adenovirus E1B 19 kDa interacting protein 2 | AY268590 | 2 |
| BCL2-like 2 | NM_004050 | 2 |
| CSE1 chromosome segregation 1-like | AF053640 | 2 |
| reticulon 4 | AF177332 | 2 |
| signal transducer and activator of transcription 1, 91 kDa | BT007241 | 2 |
| testis enhanced gene transcript (BAX inhibitor 1) | AF033095 | 2 |
| tumor necrosis factor (ligand) superfamily, member 10 | NM_003810 | 2 |
| apoptosis antagonizing transcription factor | NM_012138 | 1 |
| apoptotic protease activating factor | AF013263 | 1 |
| baculoviral IAP repeat-containing 2 | AF207599 | 1 |
| BCL2-antagonist of cell death | AF031523 | 1 |
| BCL2-antagonist/killer 1 | U16812 | 1 |
| BCL2-associated athanogene hypothetical protein HSPC177 | AF022224 | 1 |
| beclin 1 (coiled-coil, myosin-like BCL2 interacting protein) | AF077301 | 1 |
| caspase 3, apoptosis-related cysteine protease | NM_004346 | 1 |
| caspase 4, apoptosis-related cysteine protease | NM_033307 | 1 |
| caspase recruitment domain family, member 4 | AF126484 | 1 |
| cell division cycle 2-like 2 | NM_033486 | 1 |
| cell death-inducing DFFA-like effector b | AF190901 | 1 |
| chromosome 20 open reading frame 97 | AY247738 | 1 |
| cisplatin resistance-associated overexpressed protein | AB034205 | 1 |
| cullin 3 | AF052147 | 1 |
| cullin 4A | AF077188 | 1 |
| cyclin-dependent kinase inhibitor 1A (p21, Cip1) | L47232 | 1 |
| cytochrome c, somatic | BC009578 | 1 |
| DEAD (Asp-Glu-Ala-Asp) box polypeptide 41 | BT007583 | 1 |
| death associated protein 3 | NM_004632 | 1 |
| death-associated protein | NM_004394 | 1 |
| dual specificity phosphatase 22 | AK093183 | 1 |
| epithelial membrane protein 2 | NM_001424 | 1 |
| engulfment and cell motility 3 (ced-12 homolog, C. elegans) | NM_024712 | 1 |
| estrogen receptor binding site associated, antigen, 9 | NM_004215 | 1 |
| Fas (TNFRSF6)-associated via death domain | NM_003824 | 1 |
| FAST kinase | NM_025096 | 1 |
| fragile X mental retardation, autosomal homolog 1 | NM_005087 | 1 |
| growth arrest and DNA-damage-inducible, beta | AF087853 | 1 |
| histone deacetylase 3 | AF059650 | 1 |
| immediate early response 3 | NM_003897 | 1 |
| insulin-like growth factor 1 receptor | NM_000875 | 1 |
| NCK-associated protein 1 | AB014509 | 1 |
| optic atrophy 1 (autosomal dominant) | NM_015560 | 1 |
| presenilin 1 (Alzheimer disease 3) | AK094186 | 1 |
| programmed cell death 2 | NM_002598 | 1 |
| programmed cell death 6 interacting protein | AF151793 | 1 |
| protein kinase, interferon-inducible double stranded RNA dependent | M35663 | 1 |
| RAD21 homolog (S. pombe) | NM_006265 | 1 |
| RelA-associated inhibitor | BC014499 | 1 |
| SON DNA binding protein | AF380179 | 1 |
| Tax1 (human T-cell leukemia virus type I) binding protein 1 | NM_006024 | 1 |
| TIA1 cytotoxic granule-associated RNA binding protein-like 1 | NM_003252 | 1 |
| tumor necrosis factor receptor superfamily, member 10a | NM_003844 | 1 |
| tumor necrosis factor receptor superfamily, member 10b | NM_003842 | 1 |
| tumor necrosis factor receptor superfamily, member 1A | M63121 | 1 |
| tumor protein p53 (Li-Fraumeni syndrome) | AF307851 | 1 |
| tumor protein p73-like | NM_003722 | 1 |
| unc-13-like (C. elegans) | AF020202 | 1 |
| v-abl Abelson murine leukemia viral oncogene homolog 1 | X16416 | 1 |
| voltage-dependent anion channel 1 | L06132 | 1 |

Caspases are cysteine proteases that are key initiators or effectors of the apoptotic program [61]. In the KC cornea dataset, there are three clones for the effector or “executioner” caspase, caspase-6, that exerts its effects by cleavage of lamin A and other substrates [62, 63]. Of the three clones, two are essentially full-length and clearly come from the longer, α form of caspase-6. There are also single clones for another effector, caspase-3 and for caspase-4.

A Novel Gene: KC6

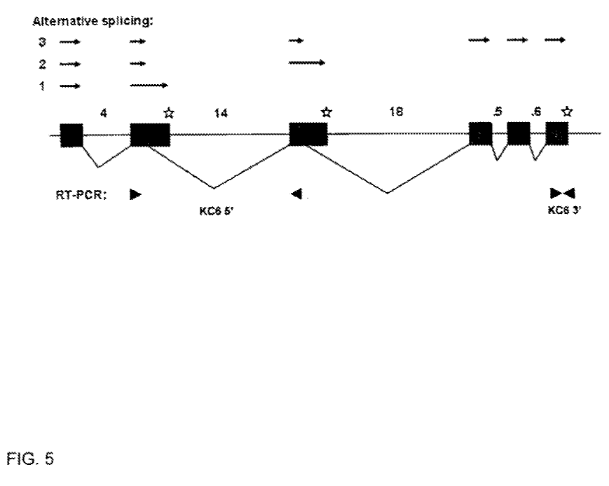

Among the abundantly expressed genes identified in the KC dataset is a novel gene of unknown function. For convenience, this is designated ‘KC6”, since a group of six ESTs from KC define the transcription unit on human chromosome 18q12.3, close to the gene for phosphoinositide-3-kinase, class 3 (PIK3C3). The only other ESTs in the current version of dbEST that appear to come from the same gene are from embryonic stem cells (accession numbers CD654078 and CN413612). The KC ESTs define a gene with at least six exons, while the partial sequence of EST CN413612 suggests the possibility of an additional upstream exon. This gene exhibits alternative splicing and alternative 3′ ends (with alternative polyadenylation signals) (FIG. 5) and has all the hallmarks of a polymerase Independent gene that encodes an mRNA, with the notable exception that there is no evidence of any significant open reading frame in the transcribed sequences. EST CN413612 from human ES cells, which may represent a partial transcript of the same gene, contains a 5′ Alu-like sequence that contains a short ORF similar to some Alu-derived sequences in GenBank, but overall there is no clear evidence that this novel gene encodes a protein and it may instead belong to the largely mysterious class of non-coding RNAs [64]. The program Repeatmasker (used to delineate repetitive sequences in the genome) detects short stretches of LINE-like sequence [65] in some parts of the KC6 gene, suggesting a possible relationship with retroviral-like sequences. No clear mouse ortholog is apparent, but examination of the mouse genome reveals the presence of four ESTs, also from ES cells, that map to an equivalent region of the mouse genome (on mouse chromosome 18), close to the orthologous PIK3C3 gene and this region of the mouse genome also contains LINE-related sequences. This is under further investigation. The sequence of the splice variant encompassing all six exons observed in the KC dataset has been submitted to GenBank (Accession number: bankit665650).

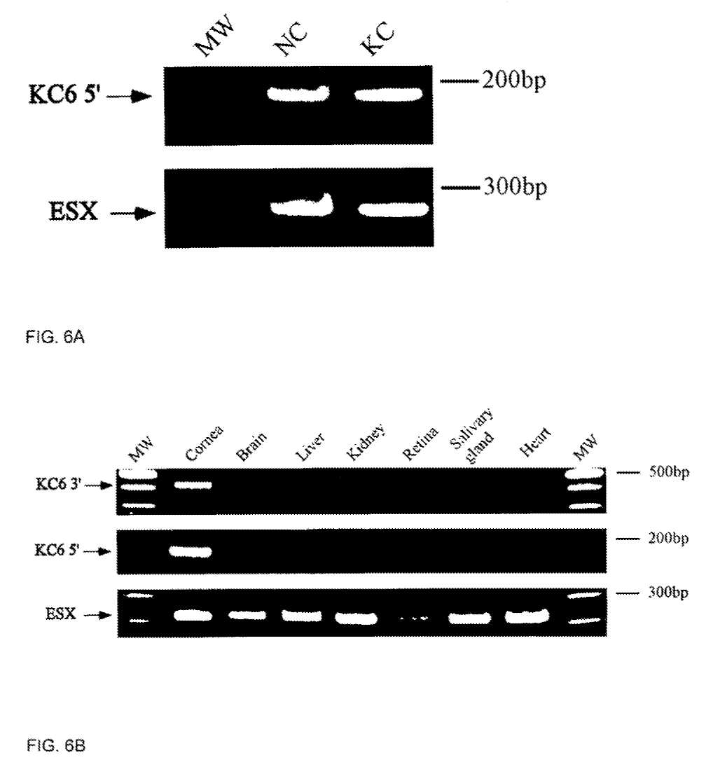

RT-PCR was used to verify the expression of KC6 in KC cornea and to examine its tissue preference (FIG. 6). Two different primer sets, one for the sixth exon defined by the identified KC6 cones and one that crosses the intron between the first and second exons defined by the KC6 ESTs, were used. These were tested on freshly obtained samples of epithelium from KC and non-KC myopic human cornea. Primers for the epithelial transcription factor ELF3/ESX were used as positive controls. While ELF3/ESX transcripts were detected at similar levels in both cornea samples and in retina, brain, kidney and heart, KC6 transcripts were detected in both KC and “format” cornea but not in any other tissue tested, suggesting that KC6 is cornea-preferred or even cornea specific.

Aquaporin 5

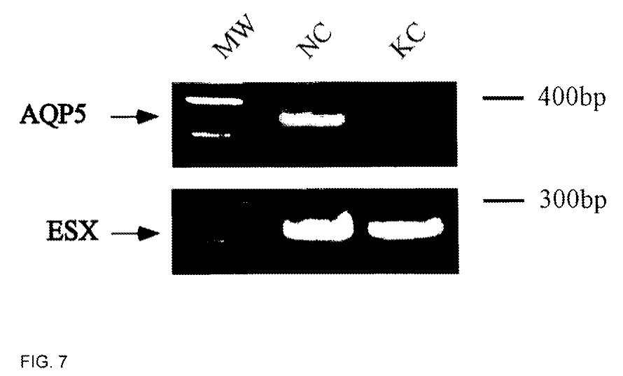

In addition to the unexpected presence of KC6, the KC EST analysis revealed an unexpected absence, Aquaporin 5 (AQP5) is a member of a large family of integral membrane proteins whose principal function is to serve as water channels [66]. AQP5 is expressed in salivary and lacrimal glands and in corneal epithelium, while the related AQP1 is expressed in corneal endothelium [67]. Water is a major component of the corneal stroma and cell layers and indeed, deletion of AQP5 in genetically modified mice causes corneal swelling [68]. AQP5 cDNA is present in the BodyMap corneal epithelium collection [10] and is also prominent in SAGE analysis of mouse cornea [13]. The human KC cornea cDNA collection contains seven clones for the endothelial marker AQP1 and one clone for AQP3, which is known to be expressed in conjunctiva [67]. However, it contains no clones for AQP5, even though many other epithelial markers are present. This striking absence was confirmed by semi-quantitative RT-PCT analysis of samples from KC and non-KC (posterior polymorphous dystrophy with normal epithelium) cornea, again using the transcription factor ELF3/ESX as a moderately abundant marker for epithelium (FIG. 7). ESX levels were indistinguishable in both KC and non-KC cornea, but while AQP5 was strongly amplified from non-KC cornea, it was barely detectable in KC. The PCR bands obtained were cloned and sequenced for conformation of their identity. Indeed, sequence of the open reading frame of AQP5 amplified from KC cornea showed no mutations and normal splicing. It seems likely that the gene for AQP5 is normal in KC but that expression of the gene is suppressed in KC, at least at the stage of disease progression at which transplantation is indicated. At this point it is unknown whether suppression of AQP5 expression is a major contributor to the phenotype of KC or whether it is one of a number of late stage downstream effects. It is possible that the genetic heterogeneity of KC reflects multiple genes in a common pathway, one of whose targets is AQP5. More detailed analysis of KC by microarray is underway to search for other affected genes.

To exert a phenotype, reduced gene expression should be associated with reduced levels of protein. Attempts were therefore made to examine protein for AQP5 in normal and KC cornea. However, from the samples at our disposal, no reactive bands for AQP5 were observed in western blot of either normal or KC, although as a control another epithelial membrane protein, connexin 50, could be detected. Several different commercial antisera were tested with the same result. All these antisera are targeted to the same C-terminal peptide of AQP5. It is known that AQP0 (the tens protein MIP) loses its C-terminal peptide with age [69], and it is possible the same thing has happened to AQP5 in the adult human corneal samples we have tested. Antibodies that target other regions of the AQP5 protein are therefore preferred.

The KC cornea cDNA library is an excellent source of clones for genes expressed in human cornea and greatly expands the representation of such genes in the databases. Further sequencing of the same library would certainly reveal even more of the transcriptional repertoire of this tissue. However the analysis to date has already identified about 4000 cornea-expressed genes and gives us a number of interesting new candidates for genes whose expression may be affected in KC. Most notable in this category is the virtual absence of AQP5 transcripts in the KC corneas. The library was made from seven pooled KC corneas and RT-PCR of an additional KC samples confirms that mRNA for this important water channel protein is barely detectable in the diseased cornea.

A water channel defect would be a plausible contributor to the stromal thinning characteristic of KC and although AQP5 is located on human chromosome 12q13, which has not been identified as a locus for KC, it is possible that the identified disease loci correspond to genes in pathways that converge on AQP5. However, deletion of AQP5 in mice produces corneal swelling [68], rather than the stromal thinning seen in human KC patients. While this could reflect a species difference in expression and behavior of aquaporins between human and rodents eye [70] it is also likely that other genes are also involved in the KC phenotype, which is more complex that a simple AQP5 null. Other genes at unexpectedly high levels in the KC dataset, such as desmogleins, or variations in expression patterns of keratins and collagens may also be markers for the disease phenotype. AQP5 and these and other genes may be downstream, possibly late, targets for the progressing disease. The abundance in the KC library of cDNAs for genes involved in apoptosis is striking and may reflect an elevation in programmed cell death associated with the disease, but again this may be effect rather than cause. The EST analysis described here provides ample candidates for further study.

Lastly, the expression of KC6 reveals an unexpected new marker for cornea. So far, this mysterious gene appears to be preferentially expressed in cornea. Since ESTs have also been obtained from embryonic stem cells, an association with corneal stem cells is likely. The corneal epithelium is known to have populations of stem cells that respond to corneal wounding and to the normal loss of epithelial cells by differentiation and replacement of the lost cells. No molecular markers for these stem cells have yet been identified [71]. They are thought be principally localized to the corneal limbus, which is missing from the KC button samples. If KC6 does have an association with stem cell populations in the cornea, it would suggest that some of these cells are more widely distributed.

### REFERENCES

### Cited in Example 3

1. Oyster, C. W., The Human Eye. Structure and Function. 1999, Sunderland, Mass.: Sinauer Associates Inc.
2. Klintworth, G. K., The molecular genetics of the corneal dystrophies—current status. Front Biosci, 2003. 8: p. d687-713.
3. Jun, A. S. and D. F. Larkin, Prospects for gene therapy in corneal disease. Eye, 2003. 17(8): p. 906-11.
4. Wistow, G., A project for ocular bioinformatics: NEIBank. Mol Vis, 2002 . 8: p. 161-3.
5. Wistow, G., et al., Expressed sequence tag analysis of adult human iris for the NEIBank Project: steroid-response factors and similarities with retinal pigment epithelium. Mol Vis, 2002. 8: p. 185-95.
6. Wistow, G., et al., Expressed sequence tag analysis of adult human lens for the NEIBank Project: over 2000 non-redundant transcripts, novel genes and splice variants. Mol Vis, 2002. 8: p. 171-84.
7. Wistow, G., et al., Expressed sequence tag analysis of human RPE/choroid for the NEIBank Project: over 6000 non-redundant transcripts, novel genes and splice variants. Mol Vis, 2002. 8: p. 205-20.
8. Wistow, G., et al., Expressed sequence tag analysis of human retina for the NEIBank Project: retbindin, an abundant, novel retinal cDNA and alternative splicing of other retina-preferred gene transcripts. Mol Vis, 2002. 8 p. 196-204
9. Tomarev, S. I., et al., Gene expression profile of the human trabecular meshwork: NEIBank sequence tag analysis. Invest Ophthalmol Vis Sci, 2003. 44(6): p. 2588-96.
10. Nishida, K., et al., A gene expression profile of human corneal epithelium and the isolation of human keratin 12 cDNA. Invest Ophthalmol Vis Sci, 1996. 37(9): p. 1800-9.
11. Sakai, R., et al., Construction of human corneal endothelial cDNA library and identification of novel active genes. Invest Ophthalmol Vis Sci, 2002. 43(6): p. 1749-56.
12. Gottsch, J. D., et al., Gene expression in donor corneal endothelium. Arch Ophthalmol, 2003. 121(2): p. 252-8.
13. Norman, B., J. Davis, and J. Piatigorsky, Postnatal gene expression in the normal mouse cornea by SAGE. Invest Ophthalmol Vis Sci, 2004. 45(2): p. 429-40.
14. Heon, E., et al., VSX1: a gene for posterior polymorphous dystrophy and keratoconus. Hum Mol Genet, 2002. 11(9): p. 1029-36.
15. Rabinowitz, Y. S., The genetics of keratoconus. Ophthalmol Clin North Am. 2003. 16(4): p. 607-20 vii.
16. Rabinowitz, Y. S., Keratoconus. Surv Ophthalmol, 1998. 42(4): p. 297-319.
17. Maguire, L. J. and W. M. Bourne, Corneal topography of early keratoconus. Am J Ophthalmol, 1989. 108(2): p. 107-12.
18. Li, X., et al., Longitudinal study of the normal eyes in unilateral keratoconus patients Ophthalmology, 2004. 111(3): p. 440-6.
19. Nielsen, K., et al., Identification of differentially expressed genes in keratoconus epithelium analyzed on microarrays. Invest Ophthalmol Vis Sci, 2003. 44(6): p. 2466-76.
20. Brancati, F., et al., A locus for autosomal dominant keratoconus maps to human chromosome 3p14-q13. J Med Genet, 2004. 41(3): p. 188-92.
21. Tang, Y. M., et al., A two-stage genome-wide scan identifies a new susceptibility locus for autosomal dominant keratoconus in a large Caucasian pedigree. Am. J. Hum. Genet., 2003. 73(sup.): p. 1822.
22. Hughes, A. E, et al., Familial keratoconus with cataract: linkage to the long arm of chromosome 15 and exclusion of candidate genes. Invest Ophthalmol Vis Sci, 2003. 44(12): p. 5063-6.
23. Tyynismaa, H., et al., A locus for autosomal dominant keratoconus: linkage to 16q22.3-q23.1 in Finnish families. Invest Ophthalmol Vis Sci, 2002. 43(10): p. 3160-4.
24. Fullerton, J., et al., Identity-by-descent approach to gene localisation in eight individuals affected by keratoconus from north-west Tasmania, Australia. Hum Genet, 2002. 110(5): p. 462-70.
25. Zhu, L. X., et al., Identification of a putative locus for keratoconus on Chromosome 21. Am. J. Hum. Genet., 1999. 65 (Suppl.): p. 161.
26. Wistow, G., et al., Grouping and identification of sequence tags (GRIST): bioinformatics tools for the NEIBank database. Mol Vis, 2002. 8: p. 164-70.
27. Kim, H. S., et al., BIGH3 gene mutations and rapid detection in Korean patients with corneal dystrophy. Cornea, 2001. 20(8): p. 844-9.
28. Li, W., et al., cDNA clone to chick corneal chondroitin/dermatan sulfate proteoglycan reveals identity to decorin. Arch Biochem Biophys, 1992. 296(1): p. 190-7.
29. Abedinia, M., et al., Bovine corneal aldehyde dehydrogenase: the major soluble corneal protein with a possible dual protective role for the eye. 1990. 51: p. 419-426.
30. Cooper, D. L., et al., Bovine corneal protein 54K (BCP54) is a homologue of the tumor-associated (class 3) rat aldehydrogenase (RATALD) gene. 1991. 98: p. 201-207.
31. Zieske, J. D., et al., à-Enolase is restricted to basal cells of stratified squamous epithelium. 1992. 151(1): p. 18-26.
32. Kinoshita, S., et al., Characteristics of the human ocular surface epithelium. Prog Retin Eye Res, 2001. 20(5): p. 639-73.
33. Liu, C. Y., et at., The cloning of mouse keratocan cDNA and genomic DNA and the characterization of its expression during eye development. J Biol Chem, 1998. 273(35); p. 22584-8.
34. Wu, H., J. R. Stanley, and G. Cotsarelis, Desmoglein isotype expression in the hair follicle and its cysts correlates with type of keratinization and degree of differentiation. J Invest Dermatol, 2003. 120(6): p. 1052-7.
35. Benz, K., et al., Molecular analysis of expansion, differentiation, and growth factor treatment of human chondrocytes identifies differentiation markers and growth-related genes. Biochem Biophys Res Commun, 2002. 293(1): p. 284-92.
36. Beardsley, T. L, and G. N. Foulks, An association of keratoconus and mitral valve prolapse. Ophthalmology, 1982. 89(1): p. 35-7.
37. Judisch, G. F., M. Wazin, and J. H. Krachmer, Ocular Ehlers-Danlos syndrome with normal lysyl hydroxylase activity. Arch Ophthalmol, 1976. 94(9); p. 1489-91.
38. Robertson, I., Keratoconus and the Ehlers-Danlos syndrome: a new aspect of keratoconus. Med J Aust, 1975. 1(18); p. 571-3.
39. Sharif, K. W., T. A. Casey, and J. Coltart, Prevalence of mitral valve prolapse in keratoconus patients. J R Soc Med, 1992. 85(8); p. 446-8.
40. Evereklioglu, C., et al., Central corneal thickness is lower in osteogenesis imperfecta and negatively correlates with the presence of blue sclera. Ophthalmic Physiol Opt, 2002. 22(6): p. 511-5.
41. Zimmermann, D. R., et al., Comparative studies of collagens in normal and keratoconus corneas. Exp Eye Res, 1988. 46(3); p. 431-42.
42. Jun, A. S., et al., Microarray analysis of gene expression in human donor corneas. Arch Ophthalmol, 2001. 119(11): p. 1629-34.
43. Ashburner, M., et at. Gene ontology: tool for the unification of biology. The Gene Ontology Consortium. Nat Genet, 2000. 25(1); p. 25-9.
44. Feo, S., et al., ENO1 gene product binds to the c-myc promoter and acts as a transcriptional repressor: relationship with Myc promoter-binding protein 1 (MBP-1). FEBS Lett, 2000. 473(1); p. 47-52.
45. Wistow, G. J., et al., Tau-crystallin/alpha-enolase: one gene encodes both an enzyme and a tens structural protein. J Cell Biol, 1988. 107(6 Pt 2): p. 2729-36.
46. Graham, C., J. Hodin, and G. Wistow, A retinaldehyde dehydrogenase as a structural protein in a mammalian eye lens. Gene recruitment of eta-crystallin. J Biol Chem, 1996. 271(26); p. 15623-8.
47. Wistow, G. and H. Kim, Lens protein expression in mammals: taxon-specificity and the recruitment of crystallins. J Mol Evol, 1991. 32(3): p. 262-9.
48. Montgomery, M. K. and M. J. McFall-Ngai, The muscle-derived lens of a squid bioluminescent organ is biochemically convergent with the ocular lens. Evidence for recruitment of aldehyde dehydrogenase as a predominant structural protein. 1992. 267(29); p. 20999-21003.
49. Zinovieva, R. D., S. I. Tomarev, and J. Piatigorsky, Aldehyde dehydrogenase-derived ê-crystallins of squid and octopus. Specialization for lens expression. 1993. 268(15): p. 11449-11455.
50. Piatigorsky, J., et al., Omega-crystallin of the scallop lens. A dimeric aldehyde dehydrogenase class ½ enzyme-crystallin. J Biol Chem, 2000. 275(52); p. 41064-73.
51. Wawersik, S., P. Purcell, and R. L. Maas, Pax6 and the genetic control of early eye development. 2000. 31: p. 15-36.
52. Jaworski, C., et al., Alternative splicing of Pax6 in bovine eye and evolutionary conservation of intron sequences. Biochem Biophys Res Commun, 1997. 240(1); p. 196-202.
53. Okladnova, O, et al., Regulation of PAX-6 gene transcription: alternate promoter usage in human brain. Brain Res Mol Brain Res, 1998. 60(2); p. 177-92.
54. Oettgen, P., et al., Isolation and characterization of a novel epithelium-specific transcription factor, ESE-1, a member of the ets family. Mol Cell Biol, 1997. 17(8): p. 4419-33.
55. Kas, K., et at., ESE-3, a novel member of an epithelium-specific ets transcription factor subfamily, demonstrates different target gene specificity from ESE-1. J Biol Chem, 2000. 275(4): p. 2986-98.
56. Widom, R. L., et al., Cloning and characterization of hcKrox, a transcriptional regulator of extracellular matrix gene expression. Gene, 1997. 198(1-2): p. 407-20.
57. Nishimura, D. Y., et al., The forkhead transcription factor gene FKHL7 is responsible for glaucoma phenotypes which map to 6p25. Nat Genet, 1998. 19(2): p. 140-7.
58. Kim, W. J., et al., Keratocyte apoptosis associated with keratoconus. Exp Eye Res, 1999. 69(5): p. 475-81.
59. Ihrie, R. A. and L. D. Attardi, Perp-etrating p53-Dependent Apoptosis. Cell Cycle, 2004. 3(3): p. 267-269.
60. Wilson, H. L., et al., Epithelial membrane proteins induce membrane blebbing and interact with the P2X7 receptor C terminus. J Biol Chem, 2002. 277(37): p. 34017-23.
61. Thornberry, N. A. and Y. Lazebnik, Caspases: enemies within. Science, 1998. 281(5381): p. 1312-6.
62. Alnemri, E. S., Mammalian cell death proteases: a family of highly conserved aspartate specific cysteine proteases. J Cell Biochem, 1997. 64(1): p. 33-42.
63. Slee, E. A., C. Adrain, and S. J. Martin, Executioner caspase-3, -6, and -7 perform distinct, non-redundant roles during the demolition phase of apoptosis. J Biol Chem, 2001. 276(10): p. 7320-6.
64. Morey, C. and P. Avner, Employment opportunities for non-coding RNAs. FEBS Lett, 2004. 567(1): p. 27-34.
65. Ostertag, E. M. and H. H. Kazazian, Jr., Biology of mammalian L1 retrotransposons. Annu Rev Genet, 2001. 35: p. 501-38.
66. Agre, P. and D. Kozono, Aquaporin water channels: molecular mechanisms for human diseases. FEBS Lett, 2003. 555(1): p. 72-8.
67. Verkman, A. S., Role of aquaporin water channels in eye function. Exp Eye Res, 2003. 76(2): p. 137-43.
68. Thiagarajah, J. R. and A. S. Verkman, Aquaporin deletion in mice reduces corneal water permeability and delays restoration of transparency after swelling. J Biol Chem, 2002. 277(21): p. 19139-44.
69. Alcala, J. and H. Maisel, Biochemistry of lens plasma membranes and cytoskeleton, in The Ocular Lens. 1985, Marcel Dekker, Inc.: New York. p. 169-222.
70. King, L. S. and P. Agre, Man is not a rodent: aquaporins in the airways. Am J Respir Cell Mol Biol, 2001. 24(3): p. 221-3.
71. Boulton, M. and J. Albon, Stem cells in the eye. Int J Biochem Cell Biol, 2004. 36(4): p. 643-57.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for detecting keratoconus in a specimen, the method comprising:
assaying a specimen of corneal epithelium for the presence of an expression product of the AQP5 gene;
whereby an absence or reduction of the AQP5 gene product is indicative of keratoconus.

2. The method of claim 1, wherein the specimen comprises isolated RNA.

3. The method of claim 2, wherein the RNA is mRNA.

4. The method of claim 1, wherein the assaying comprises polymerase chain reaction (PCR)

5. The method of claim 1, wherein the keratoconus is sub clinical.

6. The method of claim 1, further comprising assaying the specimen for a second gene product known to be present in corneal epithelium independent of the presence of keratoconus.

7. The method of claim 6, further comprising comparing the amount of expression product of the AQP5 gene to the amount of expression product of the second gene.

8. The method of claim 7, wherein the second gene is ESX-1.

9. An assay kit for detecting keratoconus in a specimen comprising:
(a) a pair of PCR primers that specifically amplify AQP5;
(b) a set of written instructions for using the primers of (a) to perform the method set forth in claim 1.

10. The assay kit of claim 9, wherein the PCR primers produce an amplicon that crosses two introns of the AQP5 gene.

11. The assay kit of claim 9, wherein the PCR primers have the nucleotide sequences

| | |
|---|---|
| TCCACTGACTCCCGCCGCACCAGC | (SEQ ID NO: 2) |
| and | |
| TCAGCGGGTGGTCAGCTCCATGGT. | (SEQ ID NO: 3) |

12. The assay kit of claim 9, further comprising a second pair of primers that specifically amplify a second gene known to be present in corneal epithelium independent of the presence of keratoconus.

13. The assay kit of claim 12, wherein the second gene is ESX-1.

14. The assay kit of claim 13, wherein the second pair of PCR primers have the nucleic acid sequences AGGGCAAGAAGAGCAAGCAC (SEQ ID NO: 4) and AACTTGTAGACGAGTCGCCG (SEQ ID NO: 5).

15. The assay kit of claim 12., wherein the second gene is KC6 (GenBank Accession No. NR_{—}002838; SEQ ID NO: 11)

16. The assay kit of claim 15, wherein the second pair of PCR primers have nucleic acid sequences selected from:

| | |
|---|---|
| AGAATCCAGGGGAAGATGAAGCAGC; | (SEQ ID NO: 6) |
| GAGGAAGCATAGGTTGAATGATCTG; | (SEQ ID NO: 7) |
| ATTGTGTATACATGGAGGTGGGATG; | (SEQ ID NO: 8) |
| AGGTCAAGCAATCTAAGCTGCATAG. | (SEQ ID NO: 9) |

17. An isolated polynucleotide having all or a biologically active portion of the nucleic acid sequence of SEQ ID NO: 11.

18. An isolated polynucleotide primer having a nucleic acid sequence selected from:

| | |
|---|---|
| AGAATCCAGGGGAAGATGAAGCAGC; | (SEQ ID NO: 6) |
| GAGGAAGCATAGGTTGAATGATCTG; | (SEQ ID NO: 7) |
| ATTGTGTATACATGGAGGTGGGATG; | (SEQ ID NO: 8) |
| and | |
| AGGTCAAGCAATCTAAGCTGCATAG. | (SEQ ID NO: 9) |


SEQUENCE LISTING (12 sequences)

SEQ ID NO: 1 - protein, 265 aa, HOMO SAPIENS
MKKEVCSVAFLKAVFAEFLATLIFVFFGLGSALKWPSALPTILQIALAFGLAIGTLAQALGPVSGGHINPAITLALLVGNQISLLRAFFYVAAQLVGAIAGAGILYGVAPLNARGNLAVNALNNNTTQGQAMVVELILTFQLALCIFASTDSRRTSPVGSPALSIGLSVTLGHLVGIYFTGCSMNPARSFGPAVVMNRFSPAHWVFWVGPIVGAVLAAILYFYLLFPNSLSLSERVAIIKGTYEPDEDWEEQREERKKTMELTTR

SEQ ID NO: 2 - DNA, 24 bp, ARTIFICIAL SEQUENCE
  SYNTHETIC PRIMER
tccactgactcccgccgcaccagc

SEQ ID NO: 3 - DNA, 24 bp, ARTIFICIAL SEQUENCE
  SYNTHETIC PRIMER
tcagcgggtggtcagctccatggt

SEQ ID NO: 4 - DNA, 20 bp, ARTIFICIAL SEQUENCE
  SYNTHETIC PRIMER
agggcaagaagagcaagcac

SEQ ID NO: 5 - DNA, 20 bp, ARTIFICIAL SEQUENCE
  SYNTHETIC PRIMER
aacttgtagacgagtcgccg

SEQ ID NO: 6 - DNA, 25 bp, ARTIFICIAL SEQUENCE
  SYNTHETIC PRIMER
agaatccaggggaagatgaagcagc

SEQ ID NO: 7 - DNA, 25 bp, ARTIFICIAL SEQUENCE
  SYNTHETIC PRIMER
gaggaagcataggttgaatgatctg

SEQ ID NO: 8 - DNA, 25 bp, ARTIFICIAL SEQUENCE
  SYNTHETIC PRIMER
attgtgtatacatggaggtgggatg

SEQ ID NO: 9 - DNA, 25 bp, ARTIFICIAL SEQUENCE
  SYNTHETIC PRIMER
aggtcaagcaatctaagctgcatag

SEQ ID NO: 10 - protein, 744 aa, HOMO SAPIENS
MNGRERHSGPTQDFNVPGLLWLQVLSVTFVTFGFRCPGCYRSCCCSGFCPSLRGPSGLNPCSLQSPTQFCLLTMTVIPPSSTMVWQLLMWTMMGTLRSSWREMVSRMLPRLVSNSWPQAIFPLQSPKVLGLQLLVLKNGSGCFLLTQCPVFCGSETSRYNGPNLVLKYDRAQKRLVNIAVDERSSPYYALRDRQGNAIGVTACDIDGDGREEIYFLNTNNAFSGVATYTDKLFKFRNNRWEDILSDEVNVARGVASLFAGRSVACVDRKGSGRYSIYIANYAYGNVGPDALIEMDPEASDLSRGILALRDVAAEAGVSKYTGGRGVSVGPILSSSASDIFCDNENGPNFLFHNRGDGTFVDAAASAGVDDPHQHGRGVALADFNRDGKVDIVYGNWNGPHRLYLQMSTHGKVRFRDIASPKFSMPSPVRTVITADFDNDQELEIFFNNIAYRSSSANRLFRVIRREHGDPLIEELNPGDALEPEGRGTGGVVTDFDGDGMLDLILSHGESMAQPLSVFRGNQGFNNNWLRVVPRTRFGAFARGAKVVLYTKKSGAHLRIIDGGSGYLCEMEPVAHFGLGKDEASSVEVTWPDGKMVSRNVASGEMNSVLEILYPRDEDTLQDPAPLECGQGFSQQENGHCMDTNECIQFPFVCPRDKPVCVNTYGSYRCRTNKKCSRGYEPNEDGTACVGTLGQSPGPRPTTPTAAAATAAAAAAAGAATAAPVLVDGDLNLGSVVKESCEPSC

SEQ ID NO: 11 - DNA, 1726 bp, HOMO SAPIENS
tggagcctgtcagctgagcaaaggagggaaactcaggatggagaactcctccccctccatccataagtggcacaccactggcctcctggtgttattaaaggaaaattggaaaagtttccagaagattaagcagaaagcttacaacttgcacaagactctcaattttccagtggctctgacagaaggataaatcttcggctgttttaactaaaatggacggtcaagttggtatccatcgtgaagctaaatgaatatctggacaggagtcagacaaagggaaggagaatccaggggaagatgaagcagctgcaggcctgtctgctaccctatatccggggaagagccagaaattgctgaccatagagagttacattgtccagcttttcctccacgaaatggagcagcaacaaaaggccagatcattcaacctatgcttcctcaagtctgataacattagcgtttctctcagatatcagttcaagtaaggagttgccaatagaagcaaagatgccaggaatcatcgtttttatttcagtgtccttacgaagacctttgagcacaatggaacgatgaggcaacattaaatattactggaatggggtatgaaagcagctactagaaaatcctgcaagaaaaatagatggagaaactgagtgctaagagtcttgccttttagctgtcaacagtttgaagatgaaacaaatctggaacagctgatttctattgtggcagctattacccatttttgttatgcatctgtctcttcccagactataaattatttaaggtgtgagtcggagacctaggcatttcttatggccagaaccttaagcaatgtctgggatatagcagatataacagactctcagctattgcgtgaattaaggaatgagcttgagaaatacttttaagtattttcttcctggaaaagagactccaactgtcttttgtacagtgcttgaaactcataccattaatcactctttgagactaaagctctgataacctgagtgaagaattggttacaaggacgttacaatgcaccatgctaccttcagccatttgtgcttctgcataaattgttttgaccgattagccaatcagtcaccaacaagacccaaaaggaatgagaatctaaacagattgtcaccatttggtgggtttgaacaccaagaggatagagcctagagcattgtggtctgctttaagacttctgagaggcaataaggtaaggtctgaaaaaccaagagtcgggataaacaagtcaattgtgtatacatggaggtgggatgaataaagaggaggaggggttgatggaagaaaactgcacccaaaattatcgtgtgtcaaaggctggctgtggagccgatcttacaacctcaaataatgtgatcatactatcactcagcataatttgtgccccccagttttgtatgtctaggctgcttaggaaattgatgaataacagaagatgccacttatcactgagatgaaccaagtcatcccagagggagtactatttgaagatgtgtggaacattctgtgaatatagaaaaggatcccataccatgattcaattcatgactgtggctagtagagatccacatttcatgttttgctatccatgggatttaaatctgctgtcactctccttcctatgcagcttagattgcttgacctaacattacaattaaagcttacataaatgctaaaaaaaaaaaaaaaa

SEQ ID NO: 12 - DNA, 795 bp, HOMO SAPIENS
atgaagaaggaggtgtgctccgtggccttcctcaaggccgtgttcgcagagttcttggccaccctcatcttcgtcttctttggcctgggctcggccctcaagtggccgtcggcgctgcctaccatcctgcagatcgcgctggcgtttggcctggccataggcacgctggcccaggccctgggacccgtgagcggcggccacatcaaccccgccatcaccctggccctcttggtgggcaaccagatctcgctgctccgggctttcttctacgtggcggcccagctggtgggcgccattgccggggctggcatcctctacggtgtggcaccgctcaatgcccggggcaatctggccgtcaacgcgctcaacaacaacacaacgcagggccaggccatggtggtggagctgattctgaccttccagctggcactctgcatcttcgcctccactgactcccgccgcaccagccctgtgggctccccagccctgtccattggcctgtctgtcaccctgggccaccttgtcggaatctacttcactggctgctccatgaacccagcccgctcttttggccctgcggtggtcatgaatcggttcagccccgctcactgggttttctgggtagggcccatcgtgggggcggtcctggctgccatcctttacttctacctgctcttccccaactccctgagcctgagtgagcgtgtggccatcatcaaaggcacgtatgagcctgacgaggactgggaggagcagcgggaagagcggaagaagaccatggagctgaccacccgc